(19) United States
(12) Patent Application Publication
(10) Pub. No.: US 2024/0327521 A1
(43) Pub. Date: Oct. 3, 2024

(54) SYNTHETIC NUCLEIC ACID ELEMENTS FOR ENHANCING CAR T CELL EFFICACY

(71) Applicant: Seattle Children's Hospital d/b/a Seattle Children's Research Institute, Seattle, WA (US)

(72) Inventors: Michael C. Jensen, Bainbridge Island, WA (US); James Rosser, Bellingham, WA (US)

(73) Assignee: Seattle Children's Hospital d/b/a Seattle Children's Research Institute, Seattle, WA (US)

(21) Appl. No.: 18/293,330

(22) PCT Filed: Jul. 27, 2022
(86) PCT No.: PCT/US2022/074190

§ 371 Date: Jan. 29, 2024

Related U.S. Application Data
(60) Provisional application No. 63/227,264, filed on Jul. 29, 2021.

(51) Int. Cl. C07K 16/28; A61K 39/00; C07K 14/55; C07K 14/705; C07K 14/725; C12N 5/0783

(52) U.S. Cl. CPC C07K 16/2827; A61K 39/4611; A61K 39/4631; A61K 39/464411; A61K 39/464412; C07K 14/55; C07K 14/7051; C07K 14/70521; C07K 14/70578; C07K 16/2803; C12N 5/0636; A61K 2239/15; A61K 2239/17; A61K 2239/22; C07K 2317/53; C07K 2317/622; C07K 2319/03; C12N 2510/00

(57) ABSTRACT

Some embodiments of the methods and compositions provided herein include methods and/or systems for increasing an activity of a cell comprising a chimeric antigen receptor (CAR), comprising use of a first nucleic acid encoding a transcription response element (TRE); and a second nucleic acid encoding a CAR, wherein the activity of the cell is increased compared to a cell lacking the first nucleic acid. In some embodiments, the increased activity of the cell is selected from: (i) survival of a subject administered the cell, wherein the subject comprises a target cell comprising an antigen, wherein the CAR is capable of specifically binding to the antigen; (ii) killing of a target cell comprising an antigen, wherein the CAR is capable of specifically binding to the antigen; and (iii) proliferation of the cell in the presence of a target cell comprising an antigen, wherein the CAR is capable of specifically binding to the antigen.

### CROSS REFERENCE TO RELATED APPLICATIONS

This application is a U.S. National Phase Patent Application based on International Patent Application No. PCT/US2022/074190 filed Jul. 27, 2022, which claims priority to U.S. Provisional Patent Application No. 63/227,264 filed Jul. 29, 2021, the contents of which are each incorporated herein by reference in their entireties.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### REFERENCE TO SEQUENCE LISTING

The present application is being filed along with a Sequence Listing in electronic format. The Sequence Listing is provided as a file entitled 44770284.xml created on Jul. 25, 2022, which is 45,831 bytes in size. The information in the electronic format of the Sequence Listing is incorporated herein by reference in its entirety.

### FIELD OF THE INVENTION

Some embodiments of the methods and compositions provided herein include methods and/or systems for increasing an activity of a cell comprising a chimeric antigen receptor (CAR), wherein the method comprises the use of a first nucleic acid encoding a transcription response element (TRE); and a second nucleic acid encoding a CAR, wherein the activity of the cell is increased compared to a cell lacking the first nucleic acid. In some embodiments, the increased activity of the cell is selected from: (i) survival of a subject administered the cell, wherein the subject comprises a target cell comprising an antigen, wherein the CAR is capable of specifically binding to the antigen; (ii) killing of a target cell comprising an antigen, wherein the CAR is capable of specifically binding to the antigen; and (iii) proliferation of the cell in the presence of a target cell comprising an antigen, wherein the CAR is capable of specifically binding to the antigen.

### BACKGROUND OF THE INVENTION

Immunotherapy using adoptive cell transfer of chimeric antigen receptor (CAR) bearing T-cells is an effective approach to treat cancer. The structure of a CAR includes an antigen binding domain, a spacer domain, a transmembrane domain, and one or more co-stimulatory activation domains. CAR T cells may be prepared from T cells obtained from a patient or from a donor. In some instances, CARs function by binding to a specific antigen on a cell surface, which causes lysis of the antigen-bearing cell. Although considerable research has focused on the design of CAR ligand binding domains, which target desired cell surface antigens with reduced toxicity, there remains a need for additional CAR T cell-mediated therapies.

### SUMMARY OF THE INVENTION

Some embodiments provided herein include a method of increasing an activity of a cell comprising a chimeric antigen receptor (CAR), comprising introducing into a cell: a first nucleic acid encoding a transcription response element (TRE); and a second nucleic acid encoding a CAR, wherein the activity of the cell is increased compared to a cell lacking the first nucleic acid.

In some embodiments, the increased activity of the cell is selected from: (i) survival of a subject administered the cell, wherein the subject comprises a target cell comprising an antigen, wherein the CAR is capable of specifically binding to the antigen; (ii) killing of a target cell comprising an antigen, wherein the CAR is capable of specifically binding to the antigen; and (iii) proliferation of the cell in the presence of a target cell comprising an antigen, wherein the CAR is capable of specifically binding to the antigen.

In some embodiments, the TRE comprises a binding site for a TRE of a gene selected from E2F1, EGR1, FOS, HIF1a, JUN, NFAT, LEF1, NFkB, SP1, PU.1, or STAT4.

In some embodiments, the first nucleic acid comprises a nucleotide sequence having at least 95% sequence identity with any one of SEQ ID NOs: 01-39. In some embodiments, the first nucleic acid comprises a nucleotide sequence having at least 95% sequence identity with SEQ ID NO:07. In some embodiments, the first nucleic acid comprises the nucleotide sequence of SEQ ID NO:07.

In some embodiments, the first nucleic acid comprises a minimal promoter linked to the TRE. In some embodiments, the minimal promoter is an IL-2 minimal promoter.

In some embodiments, a polynucleotide comprises the first nucleic acid and the second nucleic acid. In some embodiments, the first nucleic acid is 5′ of the second nucleic acid. In some embodiments, the polynucleotide comprises an insulator located between the first nucleic acid and the second nucleic acid. In some embodiments, the insulator comprises a 3′HS-A insulator. In some embodiments, a vector comprises the polynucleotide. In some embodiments, the vector is a viral vector. In some embodiments, the viral vector is selected from a lentiviral vector, and an adeno-associated viral (AAV) vector.

In some embodiments, a first vector comprises the first nucleic acid, and a second vector comprises the second nucleic acid.

In some embodiments, the CAR comprises a ligand binding domain, a spacer, a transmembrane domain, and an intracellular signaling domain. In some embodiments, the ligand binding domain comprises an antibody or antigen binding fragment thereof. In some embodiments, the ligand binding domain is single chain variable fragment. In some embodiments, the spacer comprises a portion of a hinge region of a human antibody. In some embodiments, the spacer comprises an IgG4 hinge domain. In some embodiments, the intracellular signaling domain comprises all of or a portion of CD3 zeta in combination with a costimulatory domain selected from the group consisting of CD27, CD28, 4-1BB, OX-40, CD30, CD40, PD-1, ICOS, LFA-1, CD2, CD7, NKG2C, B7-H3, and a combination thereof. In some embodiments, the intracellular signaling domain comprises a portion of CD3 zeta and a portion of 4-1BB.

Some embodiments also include stimulating the cell. In some embodiments, the stimulating comprises contacting the cell with an anti-CD3 antibody or antigen binding fragment thereof, an anti-CD28 antibody or antigen binding fragment thereof, or mixture thereof. Some embodiments also include contacting the cell with a compound selected from phorbol 12-myristate 13-acetate (PMA) or Ionomycin.

Some embodiments also include administering the cell to a subject, wherein the subject comprises a tumor cell comprising an antigen, wherein the CAR is capable of specifically binding to the antigen. In some embodiments, the administration comprises an immunotherapy.

In some embodiments, the cell is selected from a T cell, precursor T cell, or a hematopoietic stem cell. In some embodiments, the cell is a CD4+ T cell or a CD8+ T cell. In some embodiments, the cell is a CD8+ T cytotoxic lymphocyte cell selected from the group consisting of a naïve CD8+ T cell, a central memory CD8+ T cell, an effector memory CD8+ T cell, and a bulk CD8+ T cell; or a CD4+ T helper lymphocyte cell selected from the group consisting of a naïve CD4+ T cell, a central memory CD4+ T cell, an effector memory CD4+ T cell, and bulk CD4+ T cell. In some embodiments, the cell is human. In some embodiments, the cell is ex vivo.

Some embodiments provided herein include a system for increasing an activity of a cell comprising a chimeric antigen receptor (CAR), comprising: a first nucleic acid encoding a transcription response element (TRE); and a second nucleic acid encoding a CAR, wherein the activity of the cell is increased compared to a cell lacking the first nucleic acid.

In some embodiments, the increased activity of the cell is selected from: (i) survival of a subject administered the cell, wherein the subject comprises a target cell comprising an antigen, wherein the CAR is capable of specifically binding to the antigen; (ii) killing of a target cell comprising an antigen, wherein the CAR is capable of specifically binding to the antigen; and (iii) proliferation of the cell in the presence of a target cell comprising an antigen, wherein the CAR is capable of specifically binding to the antigen.

In some embodiments, the TRE comprises a binding site for a TRE of a gene selected from E2F1, EGR1, FOS, HIF1a, JUN, NFAT, LEF1, NFkB, SP1, PU.1, or STAT4.

In some embodiments, the first nucleic acid comprises a nucleotide sequence having at least 95% sequence identity with any one of SEQ ID NOs: 01-39. In some embodiments, the first nucleic acid comprises a nucleotide sequence having at least 95% sequence identity with SEQ ID NO:07. In some embodiments, the first nucleic acid comprises the nucleotide sequence of SEQ ID NO:07.

In some embodiments, the first nucleic acid comprises a minimal promoter linked to the TRE. In some embodiments, the minimal promoter is an IL-2 minimal promoter.

In some embodiments, a polynucleotide comprises the first nucleic acid and the second nucleic acid. In some embodiments, the first nucleic acid is 5′ of the second nucleic acid. In some embodiments, the polynucleotide comprises an insulator located between the first nucleic acid and the second nucleic acid. In some embodiments, the insulator comprises a 3′HS-A insulator. In some embodiments, a vector comprises the polynucleotide. In some embodiments, the vector is a viral vector. In some embodiments, the viral vector is selected from a lentiviral vector, and an adeno-associated viral (AAV) vector.

In some embodiments, a first vector comprises the first nucleic acid, and a second vector comprises the second nucleic acid.

In some embodiments, the CAR comprises a ligand binding domain, a spacer, a transmembrane domain, and an intracellular signaling domain. In some embodiments, the ligand binding domain comprises an antibody or antigen binding fragment thereof. In some embodiments, the ligand binding domain is single chain variable fragment. In some embodiments, the spacer comprises a portion of a hinge region of a human antibody. In some embodiments, the spacer comprises an IgG4 hinge domain. In some embodiments, the intracellular signaling domain comprises all of or a portion of CD3 zeta in combination with a costimulatory domain selected from the group consisting of CD27, CD28, 4-1BB, OX-40, CD30, CD40, PD-1, ICOS, LFA-1, CD2, CD7, NKG2C, B7-H3, and a combination thereof. In some embodiments, the intracellular signaling domain comprises a portion of CD3 zeta and a portion of 4-1BB.

In some embodiments, the cell is in contact with an anti-CD3 antibody or antigen binding fragment thereof, an anti-CD28 antibody or antigen binding fragment thereof, or mixture thereof.

In some embodiments, the cell is in contact with a compound selected from phorbol 12-myristate 13-acetate (PMA) or Ionomycin.

In some embodiments, the cell comprises the first nucleic acid and the second nucleic acid. In some embodiments, the cell is selected from a T cell, precursor T cell, or a hematopoietic stem cell. In some embodiments, the cell is a CD4+ T cell or a CD8+ T cell. In some embodiments, the cell is a CD8+ T cytotoxic lymphocyte cell selected from the group consisting of a naïve CD8+ T cell, a central memory CD8+ T cell, an effector memory CD8+ T cell, and a bulk CD8+ T cell; or a CD4+ T helper lymphocyte cell selected from the group consisting of a naïve CD4+ T cell, a central memory CD4+ T cell, an effector memory CD4+ T cell, and bulk CD4+ T cell. In some embodiments, the cell is human. In some embodiments, the cell is ex vivo.

### BRIEF DESCRIPTION OF THE DRAWINGS

FIG. 1 depicts a graph of probability of survival with time.

FIG. 2 depicts a graph of fluorescent signaling from Raji cells (target) expressing an mCherry reporter and normalized for each stimulation comprising a new addition of Raji cells to a co-culture with effector cells.

FIG. 3 depicts T cell expansion and includes a graph of fluorescent signaling from Raji cells (target) expressing an mCherry reporter and normalized for each stimulation comprising a new addition of Raji cells to a co-culture with effector cells.

FIG. 4 depicts a spheroid killing assay and includes a graph of fluorescent signaling from Be2 cells (target) expressing the fluorescent marker mCherry and co-cultured with effector cells.

FIGS. 5A-5C depict the cytokine output data (in pg/mL) for mock CD4+ T cells, CD19CAR CD4+ T cells, and CD19CAR CD4+ T cells with iSynPro GFP: ffluc, Ef1a. The output of cytokine IL2 (FIG. 5A), TNFa (FIG. 5B), or IFNg (FIG. 5C) was measured after each recursive stimulation with Be2-CD19t for a total of three stimulations.

FIGS. 6A-6C depict the cytokine output data (in pg/mL) for mock CD8+ T cells, CD19CAR CD8+ T cells, and CD19CAR CD8+ T cells with iSynPro GFP: ffluc. The output of cytokines IL2 (FIG. 6A), TNFa (FIG. 6B), or IFNg (FIG. 6C) was measured after each recursive stimulation with Be2-CD19t for a total of three stimulations.

### DETAILED DESCRIPTION

Some embodiments of the methods and compositions provided herein include methods and/or systems for increasing an activity of a cell comprising a chimeric antigen receptor (CAR), comprising use of a first nucleic acid encoding a transcription response element (TRE); and a second nucleic acid encoding a CAR, wherein the activity of the cell is increased compared to a cell lacking the first nucleic acid. In some embodiments, the increased activity of the cell is selected from: (i) survival of a subject administered the cell, wherein the subject comprises a target cell comprising an antigen, wherein the CAR is capable of specifically binding to the antigen; (ii) killing of a target cell comprising an antigen, wherein the CAR is capable of specifically binding to the antigen; and (iii) proliferation of the cell in the presence of a target cell comprising an antigen, wherein the CAR is capable of specifically binding to the antigen.

As disclosed herein, certain embodiments relate to the use of nucleic acid elements having activating activities, preferably in combination with nucleic acids encoding a CAR. Such nucleic acid elements include inducible synthetic promoter (iSynPro) sequences disclosed in U.S. 2020/0095573, which is hereby expressly incorporated by reference in its entirety. In some embodiments, a cell contains both a nucleic acid element having activating activities and a nucleic acid encoding a CAR. In some embodiments, a single polynucleotide comprises a nucleic acid element having activating activities and a nucleic acid encoding a CAR. In some embodiments, nucleic acid elements having activating activities increase certain activities of a cell, such as a CAR T cell, compared to a cell, such as a CAR T cell, lacking the nucleic acid elements. For example, nucleic acid elements having activating activities increase: (i) survival of a subject administered the cell, wherein the subject comprises a target cell comprising an antigen, wherein the CAR is capable of specifically binding to the antigen; (ii) killing of a target cell comprising an antigen, wherein the CAR is capable of specifically binding to the antigen; and/or (iii) proliferation of the cell in the presence of a target cell comprising an antigen, wherein the CAR is capable of specifically binding to the antigen.

A synthetic promoter library was previously generated by random ligation of transcription factor response elements (TREs), built upstream of a known IL2 minimal promoter (IL2mp) and screened by a reporter gene expression in chimeric antigen receptor (CAR) engineered T cells upon CAR activation. See U.S. 2020/0095573 which is incorporated by reference in its entirety. DNA was extracted from the sorted cells and sequenced. The top candidates of the derived promoter sequences were then synthesized and verified in the same system. The identified or selected promoters (also referred to as iSynPro) were found to be CAR activation inducible, CD3/CD28 inducible or chemical inducible (e.g. PMA/Ionomycin). For example, the promoter can be inducible by a bead comprising CD3, or CD28 or both. The iSynPro controlled gene expression was not significantly weakened even after multiple rounds of stimulation (at least four). These promoters are useful in CAR T cell therapy, such as when the expression of a desired molecule in CAR T cells is selectively needed so as to avoid a side effect seen when constitutive expression is used, for example.

In some embodiments, the iSynPro exhibits a relatively higher basal level expression. Syn-i-Pro promoters that exhibit a lower level of basal level expression can be generated by engineering a minimal promoter sequence or by changing the expression system from a lentivirus to a transposase based minicircle or nanoplasmid. One benefit of the CAR activation of gene expression of the iSynPro is that that with CAR activation, there are no drugs involved and therefore no side effects.

Some embodiments of the methods and compositions provided herein include aspects disclosed in U.S. 2020/0095573, which is hereby expressly incorporated by reference in its entirety.

### Definitions

“Conditional” or “Inducible” as used herein have their plain and ordinary meaning when read in light of the specification, and may include but is not limited to, for example, a nucleic acid construct that includes a promoter that provides for gene expression in the presence of an inducer and does not substantially provide for gene expression in the absence of the inducer. Without being limiting, examples of inducible promoters for mammalian expression constructs include tetracycline, ecdysone, streptogramins, macrolides or doxycycline inducible promoters. Without being limiting, examples of inducible promoters for bacterial expression constructs include but are not limited to a T7 promoter, lac promoter, trc promoter, tac promoter, tetA promoter, araBAD promoter or a rhaPBAD promoters. Without being limiting, insect-derived promoters include but are not limited to pB2 and polyhedrin promoters. In some alternatives herein, a promoter is provided, wherein the promoter is an inducible promoter for mammalian protein expression. In some alternatives, the promoter is an inducible synthetic promoter. In some alternatives, the promoter is selected to be activated following CAR T cell activation.

A “promoter” has its plain and ordinary meaning when read in light of the specification, and may include but is not limited to, for example, a nucleotide sequence that directs the transcription of a structural gene. In some alternatives, a promoter is located in the 5′ non-coding region of a gene, proximal to the transcriptional start site of a structural gene. Sequence elements within promoters that function in the initiation of transcription are often characterized by consensus nucleotide sequences. It is a region of DNA that initiates transcription of a particular gene. Promoters are located near the transcription start sites of genes, on the same strand and upstream on the DNA (towards the 5′ region of the sense strand). Promoters can be about 100, 200, 300, 400, 500, 600, 700, 800, or 1000 base pairs long or within a range defined by any two of the aforementioned lengths. As used herein, a promoter can be constitutively active, repressible or inducible. If a promoter is an inducible promoter, then the rate of transcription increases in response to an inducing agent. In some alternatives, the promoter is a synthetic promoter.

As used herein, “nucleic acid” or “nucleic acid molecule” have their plain and ordinary meaning when read in light of the specification, and may include but is not limited to, for example, polynucleotides or oligonucleotides such as deoxyribonucleic acid (DNA) or ribonucleic acid (RNA), oligonucleotides, fragments generated by the polymerase chain reaction (PCR), and fragments generated by any of ligation, scission, endonuclease action, exonuclease action, and by synthetic generation. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. They can also be referred to as “oligonucleotides.”

“Transcription factor response elements,” have their plain and ordinary meaning when read in light of the specification, and may include but is not limited to, for example, short sequences of DNA within a gene promoter region that are able to bind specific transcription factors and regulate transcription of genes. Under conditions of stress, a transcription activator protein binds to the response element and stimulates transcription. They may also be a short (50-1500 bp) region of DNA that can be bound by proteins (activators) to increase or promote or enhance the likelihood that transcription of a particular gene will occur or the level of transcription that takes place. These activator proteins are usually referred to as transcription factors. Enhancers are generally cis-acting, located up to 1 Mbp (1,000,000 bp) away from the gene and can be upstream or downstream from the start site, and either in the forward or backward direction. An enhancer may be located upstream or downstream of the gene it regulates. A plurality of enhancer domains may be used in some embodiments to generate greater transcription e.g., multimerized activation binding domains can be used to further enhance or increase the level of transcription. Furthermore, an enhancer doesn't need to be located near the transcription initiation site to affect transcription, as some have been found located in several hundred thousand base pairs upstream or downstream of the start site. Enhancers do not act on the promoter region itself, but are bound by activator proteins. These activator proteins interact with the mediator complex, which recruits polymerase II and the general transcription factors, which then begin transcribing the genes. Enhancers may also be found within introns. An enhancer's orientation may be reversed without affecting its function. Additionally, an enhancer may be excised and inserted elsewhere in the chromosome, and still affect gene transcription. An example of an enhancer binding domain is the TCR alpha enhancer. In some alternatives, the enhancer domain in the alternatives described herein is a TCR alpha enhancer.

“Transcriptional activator domains” or “Transcriptional activation domain” have their plain and ordinary meaning when read in light of the specification, and may include but is not limited to, for example, specific DNA sequences that can be bound by a transcription factor, in which the transcription factor can thereby control the rate of transcription of genetic information from DNA to messenger RNA. Specific transcription factors can include but is not limited to SP1, AP1, C/EBP, heat shock factor, ATF/CREB, c-Myc, Oct-1 and/or NF-1.

A “chimeric antigen receptor” (CAR) described herein, also known as chimeric T-cell receptor, has its plain and ordinary meaning when read in light of the specification, and may include but is not limited to, for example, an artificial T-cell receptor or a genetically engineered receptor, which grafts a desired specificity onto an immune effector cell. A CAR may be a synthetically designed receptor comprising a ligand binding domain of an antibody or other protein sequence that binds to a molecule associated with the disease or disorder and is linked via a spacer domain to one or more intracellular signaling domains of a T-cell or other receptors, such as a costimulatory domain. In some alternatives, a cell, such as a mammalian cell, is manufactured wherein the cell comprises a chimeric antigen receptor. These receptors can be used to graft the specificity of a monoclonal antibody or a binding portion thereof onto a T-cell, for example. In some alternatives herein, the genetically engineered cell further comprises a sequence that encodes a chimeric antigen receptor. In some alternatives, the chimeric antigen receptor is specific for a molecule on a tumor cell. A chimeric antigen receptor or an engineered cell expressing a T cell receptor can be used to target a specific tissue.

“Ligand” as described herein, refers to a substance that can form a complex with a biomolecule. By way of example and not of limitation, ligands can include substrates, proteins, small molecules, inhibitors, activators, nucleic acids and neurotransmitters. Binding can occur through intermolecular forces, for example ionic bonds, hydrogen bonds, and van der walls interactions. Ligand binding to a receptor protein can alter the three dimensional structure and determine its functional state. The strength of binding of a ligand is referred to as the binding affinity and can be determined by direct interactions and solvent effects. A ligand can be bound by a “ligand binding domain.” A ligand binding domain, for example, can refer to a conserved sequence in a structure that can bind a specific ligand or a specific epitope on a protein. The ligand binding domain or ligand binding portion can comprise an antibody or binding fragment thereof or scFv, a receptor ligand or mutants thereof, peptide, and/or polypeptide affinity molecule or binding partner. Without being limiting, a ligand binding domain can be a specific protein domain or an epitope on a protein that is specific for a ligand or ligands.

“PMA” or “phorbol 12-myristate 13-acetate” is a diester of phorbol and a potent tumor promoter often employed in biomedical research to activate the signal transduction enzyme protein kinase C (PKC). In the alternatives, herein, PMA is used to induce a inducible synthetic promoter.

“Ionomycin” is an ionophore produced by the bacterium Streptomyces conglobatus. It is used in research to raise the intracellular level of calcium (Ca2+) and as a research tool to understand Ca2+ transport across biological membranes. It is also used to stimulate the intracellular production of the following cytokines; interferon, perforin, IL-2, and/or IL-4-usually in conjunction with PMA. In the alternatives herein, the lonomycin is used to induce an inducible synthetic promoter.

A “minimal promoter” is used to get a low amount of transcription of a target gene. They have key sequences to specify the transcription start site, but only weakly activates transcription because it does not recruit RNA Polymerase or transcription factors strongly. In the alternatives herein, the minimal promoter sequence is an IL2-minimal promoter sequence which is a fragment of IL2 promoter (−70 to +47) containing a TATA box. Prior to construction and use of an IL-2 minimal promoter, a commercial minimal promoter from an inducible gene expression construct was tested. The commercially available minimal promoter was shown to work in cell lines but not in primary T cells.

A “protein” is a macromolecule comprising one or more polypeptide chains. A protein can also comprise non-peptide components, such as carbohydrate groups. Carbohydrates and other non-peptide substituents can be added to a protein by the cell in which the protein is produced, and will vary with the type of cell. Proteins are defined herein in terms of their amino acid backbone structures; substituents such as carbohydrate groups are generally not specified, but can be present nonetheless. In some alternatives, a cell is provided wherein the cell comprises a vector, wherein the vector comprises a gene encoding a protein, an antibody or binding fragment thereof, pro-proliferation molecule or molecule that can eradicate tumors.

An “antibody” as described herein refers to a large Y-shape protein produced by plasma cells that is used by the immune system to identify and neutralize foreign objects such as bacteria and viruses. The antibody protein can comprise four polypeptide chains; two identical heavy chains and two identical light chains connected by disulfide bonds. Each chain is composed of structural domains called immunoglobulin domains. These domains can contain about 70-110 amino acids and are classified into different categories according to their size and function.

“Pro-proliferation molecule,” as described herein, refer to chimeric cytokine receptors such as CCR (CD122), CCR (CD127), CCR (CD360), or caSTAT5, miRNA such as miRNA155, dnSHP1, or dnSHP2, PD1 chimeras such as PD1: MyD88, PD1: CD28, or CD200: CD28. In some alternatives herein, the inducible synthetic promoter drives the synthesis of a pro-proliferation molecule.

“Inducible synthetic promoter library, as used herein has its plain and ordinary meaning when read in light of the specification, and may include but is not limited to, for example, CAR activation inducible promoter library, T cell exhaustion inducible promoter library, tumor micro-environment inducible promoter library, or a hypoxia inducible promoter library.

“Inducible synthetic promoter” (iSynPro) as used herein has its plain and ordinary meaning when read in light of the specification, and may include but is not limited to, for example, a CAR activation inducible promoter library, T cell exhaustion inducible promoter library, tumor micro-environment inducible promoter library, or a hypoxia inducible promoter library.

“T cell precursors” as described herein refers to lymphoid precursor cells that can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8, and are therefore classed as double-negative (CD4 CD8) cells. As they progress through their development, they become double-positive thymocytes (CD4 CD8), and finally mature to single-positive (CD4 CD8 or CD4 CD8) thymocytes that are then released from the thymus to peripheral tissues.

CD19 as used herein has its plain and ordinary meaning when read in light of the specification, and may include but is not limited to, for example, a protein that is found on the surface of white blood cells and can assemble with the antigen receptor of B lymphocytes in order to decrease the threshold for antigen receptor-dependent stimulation. CD19 is expressed on follicular dendritic cells and B cells. CD19 is present on B cells from earliest recognizable B-lineage cells during development to B-cell blasts but is lost on maturation to plasma cells. CD19 primarily acts as a B cell co-receptor in conjunction with CD21 and CD81. Upon activation, the cytoplasmic tail of CD19 becomes phosphorylated, which leads to binding by Src-family kinases and recruitment of PI-3 kinase. As on T-cells, several surface molecules form the antigen receptor and form a complex on B lymphocytes.

Mutations in CD19 are associated with severe immunodeficiency syndromes characterized by diminished antibody production. For example, aberrant expression of CD19 is a marker of monocytic lineage in acute myelogenous leukemia. Since CD19 is a hallmark of B-cells, the protein can be used to diagnose cancers that arise from this type of cell, notably B-cell lymphomas. Since 2011, therapies targeting CD19 have begun to enter clinical trials. Most current experimental anti-CD19 drugs in development work by exploiting the presence of CD19 to direct the therapy specifically towards B-cell cancers. However, it is now emerging that the protein plays an active role in driving the growth of these cancers, by stabilizing the concentrations of the MYC oncoprotein. Thus, CD19 and its downstream signaling can be attractive therapeutic targets.

“Subject” or “patient,” as described herein, refers to any organism upon which the alternatives described herein may be used or administered, e.g., for experimental, diagnostic, prophylactic, and/or therapeutic purposes. Subjects or patients include, for example, animals. In some alternatives, the subject is mice, rats, rabbits, non-human primates, and/or humans. In some alternatives, the subject is a cow, sheep, pig, horse, dog, cat, primate or a human.

“Cytokines” as described herein, refers to small proteins (5-25 kDa) that are important in cell signaling. Cytokines are released by cells and affect the behavior of other cells, and sometimes the releasing cell itself, such as a T-cell. Cytokines can include, for example, chemokines, interferons, interleukins, lymphokines, and/or tumor necrosis factor. Cytokines can be produced by a broad range of cells, which can include, for example, immune cells like macrophages, B lymphocytes, T lymphocytes and/or mast cells, as well as, endothelial cells, fibroblasts, and/or various stromal cells.

Cytokines can act through receptors, and are important in the immune system as the cytokines can modulate the balance between humoral and cell-based immune responses, and they can regulate the maturation, growth, and responsiveness of particular cell populations. Some cytokines enhance or inhibit the action of other cytokines in complex ways. Without being limiting, cytokines can include, for example, Acylation stimulating protein, Adipokine, Albinterferon, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL5, CCL6, CCL7, CCL8, CCL9, Chemokine, Colony-stimulating factor, CX3CL1, CX3CR1, CXCL1, CXCL10, CXCL11, CXCL13, CXCL14, CXCL15, CXCL16, CXCL17, CXCL2, CXCL3, CXCL5, CXCL6, CXCL7, CXCL9, Erythropoietin, Gc-MAF, Granulocyte colony-stimulating factor, Granulocyte macrophage colony-stimulating factor, Hepatocyte growth factor, IL 10 family of cytokines, IL 17 family of cytokines, ILIA, ILIB, Inflammasome, Interferome, Interferon, Interferon beta 1a, Interferon beta 1b, Interferon gamma, Interferon type I, Interferon type II, Interferon type III, Interleukin, Interleukin 1 family, Interleukin 1 receptor antagonist, Interleukin 10, Interleukin 12, Interleukin 12 subunit beta, Interleukin 13, Interleukin 15, Interleukin 16, Interleukin 2, Interleukin 23, Interleukin 23 subunit alpha, Interleukin 34, Interleukin 35, Interleukin 6, Interleukin 7, Interleukin 8, Interleukin 36, Leukemia inhibitory factor, Leukocyte-promoting factor, Lymphokine, Lymphotoxin, Lymphotoxin alpha, Lymphotoxin beta, Macrophage colony-stimulating factor, Macrophage inflammatory protein, Macrophage-activating factor, Monokine, Myokine, Myonectin, Nicotinamide phosphoribosyltransferase, Oncostatin M, Oprelvekin, Platelet factor 4, Proinflammatory cytokine, Promegapoietin, RANKL, Stromal cell-derived factor 1, Talimogene laherparepvec, Tumor necrosis factor alpha, Tumor necrosis factors, XCL1, XCL2, GM-CSF, and/or XCR1.

“Interleukins” or IL as described herein, are cytokines that the immune system depends largely upon. Examples of interleukins, which can be utilized herein, for example, include IL-1, IL-2, IL-3, IL-4, IL-5, IL-6, 11-7, IL-8/CXCL8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and/or IL-36. Contacting T-cells with interleukins can have effects that promote, support, induce, or improve engraftment fitness of the cells. IL-1, for example can function in the maturation & proliferation of T-cells. IL-2, for example, can stimulate growth and differentiation of T-cell response. IL-3, for example, can promote differentiation and proliferation of myeloid progenitor cells. IL-4, for example, can promote proliferation and differentiation. IL-7, for example, can promote differentiation and/or proliferation of lymphoid progenitor cells, involved in B, T, and NK cell survival, development, and/or homeostasis. IL-15, for example, can induce production of natural killer cells. IL-21, for example, co-stimulates activation and/or proliferation of CD8+ T-cells, augments NK cytotoxicity, augments CD40-driven B cell proliferation, differentiation and/or isotype switching, and/or promotes differentiation of Th17 cells.

“Vector,” “Expression vector” or “construct” is a nucleic acid used to introduce heterologous nucleic acids into a cell that has regulatory elements to provide expression of the heterologous nucleic acids in the cell. Vectors include but are not limited to plasmid, minicircles, yeast, and/or viral genomes. In some alternatives, the vectors are plasmid, minicircles, yeast, or viral genomes. In some alternatives, the vector is a viral vector. In some alternatives, the viral vector is a lentivirus. In some alternatives, the vector is for protein expression in a bacterial system such as E. coli. In some alternatives, the vector is a lentiviral vector. In some alternatives, the vector is a foamy viral vector, adenoviral vectors, retroviral vectors or lentiviral vectors. In some alternatives, the vector is for protein expression in a bacterial system, such as E. coli. In some alternatives, wherein the vector is a lentiviral vector, a transposase based minicircle or a nanoplasmid.

“Combination therapy” as described herein, refers to a therapy that uses more than one medication or modality for a therapeutic application. Combination therapy, for example can also refer to multiple therapies to treat a single disease, and often all the therapies are pharmaceutical product combinations. Combination therapy can also involve prescribing and administering separate drugs in which the dosage can also have more than one active ingredient. In some alternative, a combination therapy is provided. In some alternatives, the combination therapy can further comprise administering a CAR bearing T-cell to a subject in need e.g., a human.

“Chemotherapeutic drugs” are category of anti-cancer medicaments that can use, for example, chemical substances, such as anti-cancer drugs (chemotherapeutic agents) that can be given as part of a standardized chemotherapy regimen. Chemotherapeutic drugs can be given with a curative intent, or it can aim to prolong life or to reduce symptoms (palliative chemotherapy). Additional chemotherapies can also include hormonal therapy and targeted therapy, as it is one of the major categories of medical oncology (pharmacotherapy for cancer). These modalities are often used in conjunction with other cancer therapies, such as radiation therapy, surgery, and/or hyperthermia therapy. In few cases, cancer has been known to spread due to surgery. In some alternatives, a genetically modified immune cell is administered to the tumor site prior to or after a surgical procedure.

Some newer anticancer drugs (for example, various monoclonal antibodies, humanized versions thereof and/or binding fragments thereof) are not indiscriminately cytotoxic, but rather target proteins that are abnormally expressed in cancer cells and that are essential for their growth. Such therapies are often referred to as targeted therapy (as distinct from classic chemotherapy) and are often used alongside traditional chemotherapeutic agents in antineoplastic protocols. In some alternatives, the methods described herein can further comprise administering any one or more of these targeted anti-cancer therapies (for example, various monoclonal antibodies, humanized versions thereof and/or binding fragments thereof).

Chemotherapy, in which chemotherapeutic drugs are administered, can use one drug at a time (single-agent chemotherapy) or several drugs at once (combination chemotherapy or polychemotherapy). The combination of chemotherapy and radiotherapy is chemoradiotherapy. Chemotherapy using drugs that convert to cytotoxic activity only upon light exposure is called photochemotherapy or photodynamic therapy. In some alternatives of administering the cell described herein, the method can further comprise administering to a subject having cancer, photochemotherapy or photodynamic therapy after receiving the cells.

Chemotherapeutic drugs can include but are not limited to antibody-drug conjugates (for example, an antibody or binding fragment thereof attached to a drug by a linker), nanoparticles (for example a nanoparticle can be 1-1000 nanometer sized particle for promoting tumor selectivity and aid in delivering low-solubility drugs), electochemotherapy, alkylating agents, antimetabolites (for example, 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, and/or Thioguanine), anti-tumor antibiotics, topoisomerase inhibitors, mitotic inhibitors, corticosteroids, DNA intercalating agents, or checkpoint inhibitors (for example checkpoint kinases CHK1, or CHK2). In some alternatives of the methods described herein, the genetically modified immune cells or compositions comprising genetically modified immune cells are administered in combination with one or more anti-cancer agents, such as any one or more of the foregoing compounds or therapies. In some alternatives, the one or more anti-cancer agents that are co-administered or administered in conjunction with the genetically modified immune cells, comprises antibody-drug conjugates, nanoparticles, electrochemotherapy, alkylating agents, antimetabolites, anti-tumor antibiotics, topoisomerase inhibitors, mitotic inhibitors, corticosteroids, DNA intercalating agents, or checkpoint inhibitors. In some alternatives, the antimetabolites comprises 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, or Thioguanine.

“Cancer” as described herein, can refer to a malignant tumor or a malignant neoplasma in which they involve abnormal cell growth with the potential to invade or spread to other parts of a body. In some alternatives, a method of treating, ameliorating, or inhibiting a disease or an infection in a subject is provided, wherein the method comprises delivering a cell of manufactured by any of the alternatives described herein to the subject. In some alternatives, the subject suffers from a cancer. In some alternatives, the subject is selected for a cancer therapy. In some alternatives the cancer comprises adrenal cancer, bile duct cancer, bladder cancer, bone cancer, brain cancer, breast cancer, Castleman disease, cervical cancer, colon cancer, endometrial cancer, esophagus cancer, Ewing family of tumors, eye cancer, gallbladder cancer, gastrointestinal carcinoid tumors, Hodgkin disease, Kaposi Sarcoma, kidney cancer, Laryngeal and hypopharyngeal cancer, leukemia, liver cancer, lung cancer, lymphoma, multiple myeloma, malignant mesothelioma, myelodysplastic syndrome, nasopharyngeal cancer, osteosarcoma, ovarian cancer, pancreatic cancer, pituitary tumors, prostate cancer, retinoblastoma, skin cancer, small intestine cancer, stomach cancer, testicular cancer, thymus cancer, thyroid cancer or uterine sarcoma.

### Inducible Synthetic Promoters

Certain embodiments of the methods and compositions provided herein include the use of nucleic acid elements having activating activities. Such nucleic acid elements having activating activities include inducible synthetic promoter (iSynPro) sequences disclosed in U.S. 2020/0095573 which is incorporated by reference in its entirety. TABLE 1 lists certain nucleic acid elements having activating activities.

TABLE 1

| SEQ ID NO | |
|---|---|
| Feature | Sequence |
| SEQ ID NO: 1 | CTGCTTAGGGTTAGGCGTTTTGCGCTGCTTCGCGATCGAGGA |
| S1-17 | GGAAAAACTCGATGTGACTCATTCGAAGATCAAAGGGTCGA |
| | GGAAAGTCCCCTCGACCCTTTGATCTTCGAATGACATCATCTT |
| | TCGAGGAAAGTCCCCTCGAGGGGACTTTCCTCGAGGGGACTT |
| | TCCTCGAATGAGTCACATCGATCTCCGCCCCCTCTTCGAGCGC |
| | CAAATCGAGGACGTGATCGAGTAGAGTCTAGACTCTACATTT |
| | TGACACCCCCAT |
| SEQ ID NO: 2 | CTGCTTAGGGTTAGGCGTTTTGCGCTGCTTCGCGATCGAGGG |
| S1-37 | GGCGGGGTCGATCTCCGCCCCCTCTTCGAGTTTTTCCTCCTCG |
| | AGGGGACTTTCCTCGAATGACATCATCTTTCGACCCTTTGATC |
| | TTCGAAGGAAGTTCGATCACGTCCTCGATTTGGCGCTCGAGG |
| | GGACTTTCCTCGAAGATCAAAGGGTCGAGTAGAGTCTAGACT |
| | CTACATTTTGACACCCCCAT |
| SEQ ID NO: 3 | CTGCTTAGGGTTAGGCGTTTTGCGCTGCTTCGCGATCGATTTC |
| S1-4 | CAAGAAATCGAGGAAAGTCCCCTCGACCCTTTGATCTTCGAG |
| | GAGGAAAAACTCGAATGAGTCACATCGACCCTTTGATCTTCG |
| | AAGAGGGGGCGGAGATCGATCTCCGCCCCCTCTTCGAGGAAA |
| | GTCCCCTCGACCCTTTGATCTTCGACCCCGCCCCCTCGAGGAA |
| | AGTCCCCTCGAGTAGAGTCTAGACTCTACATTTTGACACCCCC |
| | AT |
| SEQ ID NO: 4 | CTGCTTAGGGTTAGGCGTTTTGCGCTGCTTCGCGATCGATCTC |
| S1-1 | CGCCCCCTCTTCGAAGATCAAAGGGTCGAGGGGACTTTCCTC |
| | GATTTCTTGGAAATCGAATGACATCATCTTTCGATTTGGCGCT |
| | CGAATGACATCATCTTTCGACCCTTTGATCTTCGATGTGACTC |
| | ATTCGAGGGGACTTTCCTCGAAGATCAAAGGGTCGAGGGGAC |
| | TTTCCTCGAGTAGAGTCTAGACTCTACATTTTGACACCCCCAT |
| SEQ ID NO: 5 | CTGCTTAGGGTTAGGCGTTTTGCGCTGCTTCGCGATCGAGGG |
| S1-3 | GACTTTCCTCGAGGGGGCGGGGTCGATTTGGCGCTCGATCTC |
| | CGCCCCCTCTTCGAATGAGTCACATCGAGGAAAGTCCCCTCG |
| | AGGAGGAAAAACTCGATGTGACTCATTCGATTTCCAAGAAAT |
| | CGAGTAGAGTCTAGACTCTACATTTTGACACCCCCAT |
| SEQ ID NO: 6 | CTGCTTAGGGTTAGGCGTTTTGCGCTGCTTCGCGATCGATTTC |
| S1-42 | CAAGAAATCGATCTCCGCCCCCTCTTCGAATGACATCATCTTT |
| | CGAATGACATCATCTTTCGAGGAGGAAAAACTCGACCCCGCC |
| | CCCTCGATTTCTTGGAAATCGAGGAAAGTCCCCTCGAGCGCC |
| | AAATCGAAGGAAGTTCGAATGACATCATCTTTCGAATGAGTC |
| | ACATCGAGGAAAGTCCCCTCGAGGAGGAAAAACTCGAGTAG |
| | AGTCTAGACTCTACATTTTGACACCCCCAT |
| SEQ ID NO: 7 | CTGCTTAGGGTTAGGCGTTTTGCGCTGCTTCGCGATCGAATGA |
| S1-61 | GTCACATCGATCTCCGCCCCCTCTTCGAGGGGGCGGGGTCGA |
| | GGAGGAAAAACTCGAATGAGTCACATCGACCCTTTGATCTTC |
| | GAGGGGACTTTCCGGGGTGGAGCAAGCGTGACAAGTCCACG |
| | TATGACCCGACCGACGATATCGAAGCCTACGCGCTGAACGCC |
| | AGCCCCGATCGACCCCGCCCCCTCGATTTCCAAGAAATCGAA |
| | TGACATCATCTTTCGAATGACATCATCTTTCGAGGGGACTTTC |
| | CTCGAACTTCCTTCGAGGGGACTTTCCTCGAGGGGACTTTCCT |
| | CGAGGAGGAAAAACTCGAGTAGAGTCTAGACTCTACATTTTG |
| | ACACCCCCAT |
| SEQ ID NO: 8 | CTGCTTAGGGTTAGGCGTTTTGCGCTGCTTCGCGATCGATTTC |
| S1-62 | CAAGAAATCGAGTTTTTCCTCCTCGAGGGGGCGGGGTCGAGG |
| | GGACTTTCCTCGACCCTTTGATCTTCGAGGAAAGTCCCCTCGA |
| | GCGCCAAATCGATCTCCGCCCCCTCTTCGAGTAGAGTCTAGA |
| | CTCTACATTTTGACACCCCCAT |
| SEQ ID NO: 9 | CTGCTTAGGGTTAGGCGTTTTGCGCTGCTTCGCGATCGAAGAT |
| S1-15 | CAAAGGGTCGATTTCTTGGAAATCGATGTGACTCATTCGATC |
| | ACGTCCTCGAGGAGGAAAAACTCGAGGAAAGTCCCCTCGAA |
| | CTTCCTTCGAGGGGGCGGGGTCGAATGAGTCACATCGAGGAA |
| | AGTCCCCTCGAGGGGACTTTCCTCGATTTCTTGGAAATCGAA |
| | GAGGGGGCGGAGATCGAGTTTTTCCTCCTCGAGGAAAGTCCC |
| | CTCGATCGACTCTACATTTTGACACCCCCAT |
| SEQ ID NO: 10 | CTGCTTAGGGTTAGGCGTTTTGCGCTGCTTCGCGATCGAATGA |
| S1-2 | GTCACATCGAAGATCAAAGGGTCGACCCTTTGATCTTCGATT |
| | TGGCGCTCGATGTGACTCATTCGACCCCGCCCCCTCGAGGAA |
| | AGTCCCCTCGAGGAAAGTCCCCTCGAGGAAAGTCCCCTCGAG |
| | GAAAGTCCCCTCGAGTTTTTCCTCCTCGAAGAGGGGGCGGAG |
| | ATCGATTTGGCGCTCGAGGACGTGATCGAGTAGAGTCTAGAC |
| | TCTACATTTTGACACCCCCAT |
| SEQ ID NO: 11 | CTGCTTAGGGTTAGGCGTTTTGCGCTGCTTCGCGATCGATTTC |
| S1-27 | CAAGAAATCGAGTTTTTCCTCCTCGATTTCCAAGAAATCGAG |
| | GGGACTTTCCTCGAGGGGACTTTCCTCGAATGACATCATCTTT |
| | CGATGTGACTCATTCGAAGATCAAAGGGTCGAAGGAAGTTCG |
| | AATGAGTCACATCGAATGAGTCACATCGACCCTTGATCTTCG |
| | AGGAGGAAAAACTCGATTTGGCGCTCGATTTGGCGCTCGAGT |
| | AGAGTCTAGACTCTACATTTTGACACCCCCAT |
| SEQ ID NO: 12 | CTGCTTAGGGTTAGGCGTTTTGCGCTGCTTCGCGATCGATTTC |
| S1-8 | TTGGAAATCGATTTGGCGCTCGAAGGAAGTTCGAGGGGGCGG |
| | GGTCGATTTCCAAGAAATCGAGTTTTTCCTCCTCGAGCGCCA |
| | AATCGACCCTTTGATCTTCGATCACGTCCTCGAGCGCCAAATC |
| | GAGGACGTGATCGAATGAGTCACATCGAATGAGTCACATCGA |
| | ATGAGTCACATCGATTTGGCGCTCGATCGGGGGGCGGGGTCG |
| | AGGAAAGTCCCCTCGAGGAAAGTCCCCTCGATTTCCAAGAAA |
| | TCGATTTGGCGCTCGATCTCCGCCCCCTCTTCGATCTCCGCCC |
| | CCTCTTCGAGTTTTTCCTCCTCGAGTAGAGTCTAGACTCTACA |
| | TTTTGACACCCCCAT |
| SEQ ID NO: 13 | CTGCTTAGGGTTAGGCGTTTTGCGCTGCTTCGCGATCGAATGA |
| S1-30 | CATCATCTTTCGAGGAAAGTCCCCTCGACCCTTTGATCTTCGA |
| | CCCCGCCCCCTCGAGTTTTTCCTCCTCGAGGAAAGTCCCCTCG |
| | ACCCTTTGATCTTCGAGGGGACTTTCCTCGAGGGGGCGGGGT |
| | CGAGGACGTGATCGAGGAAAGTCCCCTCGAGTAGAGTCTAG |
| | ACTCTACATTTTGACACCCCCAT |
| SEQ ID NO: 14 | CTGCTTAGGGTTAGGCGTTTTGCGCTGCTTCGCGATCGAAGAT |
| S1-33 | CAAAGGGTCGAGGAGGAAAAACTCGAGGGGACTTTCCTCGA |
| | CCCTTTGATCTTCGAATGAGTCACATCGATTTCTTGGAAATCG |
| | AAGGAAGTTCGAGGGGACTTTCCTCGAGTTTTTCCTCCTCGA |
| | ACTTCCTTCGAGGAAAGTCCCCTCGAAGATCAAAGGGTCGAA |
| | GGAAGTTCGAAAGATGATGTCATTCGATTTCTTGGAAATCGA |
| | AGATCAAAGGGTCGAAGAGGGGGCGGAGATCGATTTCCAAG |
| | AAATCGAGTAGAGTCTAGACTCTACATTTTGACACCCCCAT |
| SEQ ID NO: 15 | CTGCTTAGGGTTAGGCGTTTTGCGCTGCTTCGCGATCGAATGA |
| S1-41 | GTCACATCGAGCGCCAAATCGAGGGGACTTTCCTCGAGGGGG |
| | CGGGGTCGAGTTTTTCCTCCTCGATTTCCAAGAAATCGAGGG |
| | GACTTTCCTCGAGGGGACTTTCCTCGAGGGGACTTTCCTCGA |
| | GTAGAGTCTAGACTCTACATTTTGACACCCCCAT |
| SEQ ID NO: 16 | CTGCTTAGGGTTAGGCGTTTTGCGCTGCTTCGCGATCGAGTTT |
| S1-59 | TTCCTCCTCGAGGGGACTTTCCTCGAAAGATGATGTCATTCGA |
| | TCTCCGCCCCCTCTTCGAGGGGACTTTCCTCGAGGAAAGTCCC |
| | CTCGAGGAAAGTCCCCTCGAAGATCAAAGGGTCGAATGAGTC |
| | ACATCGACCCTTTGATCTTCGACCCTTTGATCTTCGATTTCCA |
| | AGAAATCGAGGAAAGTCCCCTCGAGGAAAGTCCCCTCGAGT |
| | AGAGTCTAGACTCTACATTTTGACACCCCCAT |
| SEQ ID NO: 17 | CTGCTTAGGGTTAGGCGTTTTGCGCTGCTTCGCGATCGAGGG |
| S1-66 | GACTTTCCTCGAGGAAAGTCCCCTCGATTTCTTGGAAATCGAT |
| | TTGGCGCTCGAGGAGGAAAAACTCGATGTGACTCATTCGACC |
| | CTTTGATCTTCGAGGGGACTTTCCTCGAGGGGACTTTCCTCGA |
| | GCGCCAAATCGAGGAGGAAAAACTCGAGGGGACTTTCCTCG |
| | ATTTCTTGGAAATCGAAAGATGATGTCATTCGACCCTTTGATC |
| | TTCGAGTAGAGTCTAGACTCTACATTTTGACACCCCCAT |
| SEQ ID NO: 18 | CTGCTTAGGGTTAGGCGTTTTGCGCTGCTTCGCGATCGAGGA |
| S1-71 | AAGTCCCCTCGAGGAAAGTCCCCTCGATTTCTTGGAAATCGA |
| | ATGACATCATCTTTCGATCACGTCCTCGAGGAAAGTCCCCTC |
| | GAGTTTTTCCTCCTCGAGGGGACTTTCCTCGATTTCCAAGAAA |
| | TCGATTTCTTGGAAATCGACCCTTTGATCTTCGAGGAGGAAA |
| | AACTCGAGTAGAGTCTAGACTCTACATTTTGACACCCCCAT |
| SEQ ID NO: 19 | CTGCTTAGGGTTAGGCGTTTTGCGCTGCTTCGCGATCGATGTG |
| S1-56 | ACTCATTCGAGGAAAGTCCCCTCGATTTGGCGCTCGATGTGA |
| | CTCATTCGATTTGGCGCTCGAGGGGACTTTCCTCGATTTCCAA |
| | GAAATCGAGGGGACTTTCCTCGAGGAAAGTCCCCTCGAGTTT |
| | TTCCTCCTCGAGTAGAGTCTAGACTCTACATTTTGACACCCCC |
| | AT |
| SEQ ID NO: 20 | CTGCTTAGGGTTAGGCGTTTTGCGCTGCTTCGCGATCGATGTG |
| S1-6 | ACTCATTCGAGCGCCAAATCGATTTCCAAGAAATCGAATGAG |
| | TCACATCGAATGACATCATCTTTCGATGTGACTCATTCGAGG |
| | AAAGTCCCCTCGATTTCTTGGAAATCGAGGAAAGTCCCCTCG |
| | AGTTTTTCCTCCTCGAAGAGGGGGCGGAGATCGAATGACATC |
| | ATCTTTCGAGTAGAGTCTAGACTCTACATTTTGACACCCCCAT |
| SEQ ID NO: 21 | CTGCTTAGGGTTAGGCGTTTTGCGCTGCTTCGCGATCGAGGG |
| S1-60 | GACTTTCCTCGATCTCCGCCCCCTCTTCGAGGAAAGTCCCCTC |
| | GAAGGAAGTTCGAACTTCCTTCGAGGGGACTTTCCTCGAAGG |
| | AAGTTCGAGCGCCAAATCGAATGAGTCACATCGATTTCCAAG |
| | AAATCGATCTCCGCCCCCTCTTCGAGGAGGAAAAACTCGAGG |
| | GGACTTTCCTCGATTTCCAAGAAATCGAGGGGACTTTCCTCG |
| | ATCACGTCCTCGAGGACGTGATCGAATGAGTCACATCGATGT |
| | GACTCATTCGACCCTTTGATCTTCGAGTAGAGTCTAGACTCTA |
| | CATTTTGACACCCCCAT |
| SEQ ID NO: 22 | CTGCTTAGGGTTAGGCGTTTTGCGCTGCTTCGCGATCGATTTC |
| S1-86 | CAAGAAATCGAGGGGGCGGGGTCGAGGGGACTTTCCTCGAG |
| | GGGACTTTCCTCGAGGAAAGTCCCCTCGATCTCCGCCCCCTCT |
| | TCGAGGAAAGTCCCCTCGAACTTCCTTCGAGGAAAGTCCCCT |
| | CGAATGAGTCACATCGATTTGGCGCTCGAGGAAAGTCCCCTC |
| | GAGGGGACTTTCCTCGAGGAAAGTCCCCTCGAGGGGGGGG |
| | GTCGAACTTCCTTCGAGTAGAGTCTAGACTCTACATTTTGACA |
| | CCCCCAT |
| SEQ ID NO: 23 | CTGCTTAGGGTTAGGCGTTTTGCGCTGCTTCGCGATCGAATGA |
| S1-32 | GTCACATCGATCTCCGCCCCCTCTTCGATTTGGCGCTCGAGGG |
| | GACTTTCCTCGACCCTTTGATCTTCGAGGGGACTTTCCTCGAG |
| | GAAAGTCCCCTCGAGGAAAGTCCCCTCGAGTAGAGTCTAGAC |
| | TCTACATTTTGACACCCCCAT |
| SEQ ID NO: 24 | CTGCTTAGGGTTAGGCGTTTTGCGCTGCTTCGCGATCGATTTG |
| S1-10 | GCGCTCGATGTGACTCATTCGAGGAAAGTCCCCTCGAGGGGA |
| | CTTTCCTCGAAGATCAAAGGGTCGAAAGATGATGTCATTCGA |
| | GGAAAGTCCCCTCGACCCCGCCCCCTCGATTTCTTGGAAATC |
| | GAATGAGTCACATCGATTTCTTGGAAATCGATGTGACTCATT |
| | CGAGTAGAGTCTAGACTCTACATTTTGACACCCCCAT |
| SEQ ID NO: 25 | CTGCTTAGGGTTAGGCGTTTTGCGCTGCTTCGCGATCGAGGG |
| S1-18 | GACTTTCCTCGATGTGACTCATTCGATTTCTTGGAAATCGATT |
| | TCCAAGAAATCGACCCCGCCCCCTCGAGGAGGAAAAACTCG |
| | ATTTCCAAGAAATCGAATGACATCATCTTTCGAGGGGACTTT |
| | CCTCGAAGATCAAAGGGTCGAAGATCAAAGGGTCGAGGAGG |
| | AAAAACTCGATTTCCAAGAAATCGAATGAGTCACATCGAGTT |
| | TTTCCTCCTCGATTTCTTGGAAATCGAAGAGGGGGCGGAGAT |
| | CGATTTGGCGCTCGAGTAGAGTCTAGACTCTACATTTTGACA |
| | CCCCCAT |
| SEQ ID NO: 26 | CTGCTTAGGGTTAGGCGTTTTGCGCTGCTTCGCGATCGAAAG |
| S1-14 | ATGATGTCATTCGAATGAGTCACATCGATTTGGCGCTCGAGG |
| | GGGCGGGGTCGATTTGGCGCTCGAAAGATGATGTCATTCGAG |
| | TTTTTCCTCCTCGACCCTTTGATCTTCGAAGAGGGGGCGGAGA |
| | TCGAAAGATGATGTCATTCGAATGAGTCACATCGATTTCCAA |
| | GAAATCGATTTCCAAGAAATCGAGTAGAGTCTAGACTCTACA |
| | TTTTGACACCCCCAT |
| SEQ ID NO: 27 | CTGCTTAGGGTTAGGCGTTTTGCGCTGCTTCGCGATCGAGGA |
| S1-16 | GGAAAAACTCGATTTCTTGGAAATCGAGGGGACTTTCCTCGA |
| | AAGATGATGTCATTCGAAGATCAAAGGGTCGATGTGACTCAT |
| | TCGAGGGGACTTTCCTCGAGGGGGCGGGGTCGAATGACATCA |
| | TCTTTCGAGGGGACTTTCCTCGAGGGGACTTTCCTCGAATGAC |
| | ATCATCTTTCGATTTCTTGGAAATCGAGTAGAGTCTAGACTCT |
| | ACATTTTGACACCCCCAT |
| SEQ ID NO: 28 | CTGCTTAGGGTTAGGCGTTTTGCGCTGCTTCGCGATCGATCTC |
| S1-19 | CGCCCCCTCTTCGAGTTTTTCCTCCTCGATTTCTTGGAAATCG |
| | AAGATCAAAGGGTCGATTTCTTGGAAATCGAGGGGACTTTCC |
| | TCGAGGAAAGTCCCCTCGAGGGGACTTTCCTCGAGGAGGAAA |
| | AACTCGATTTGGCGCTCGAGTAGAGTCTAGACTCTACATTTTG |
| | ACACCCCCAT |
| SEQ ID NO: 29 | CTGCTTAGGGTTAGGCGTTTTGCGCTGCTTCGCGATCGAAAG |
| S1-26 | ATGATGTCATTCGAGTTTTTCCTCCTCGAAGGAAGTTCGAGG |
| | ACGTGATCGAAGAGGGGGCGGAGATCGAATGAGTCACATCG |
| | AAGGAAGTTCGAGGAAAGTCCCCTCGAGGAAAGTCCCCTCG |
| | AAGATCAAAGGGTCGAGTAGAGTCTAGACTCTACATTTTGAC |
| | ACCCCCAT |
| SEQ ID NO: 30 | CTGCTTAGGGTTAGGCGTTTTGCGCTGCTTCGCGATCGACCCC |
| S1-65 | GCCCCCTCGAGCGCCAAATCGAGGGGACTTTCCTCGAGGGGA |
| | CTTTCCTCGAGGGGACTTTCCTCGAGTAGAGTCTAGACTCTAC |
| | ATTTTGACACCCCCAT |
| SEQ ID NO: 31 | CTGCTTAGGGTTAGGCGTTTTGCGCTGCTTCGCGATCGAGGG |
| S2-n1 | GGCGGGGTCGAATGACATCATCTTTCGAGGAAAGTCCCCTCG |
| | AGGAAAGTCCCCTCGAGGAAAGTCCCCTCGATTTCCAAGAAA |
| | TCGACCCTTTGATCTTCGAAGATCAAAGGGTCGATGTGACTC |
| | ATTCGAAGATCAAAGGGTCGATTTGGCGCTCGAGTAGAGTCT |
| | AGACTCTACATTTTGACACCCCCAT |
| SEQ ID NO: 32 | CTGCTTAGGGTTAGGCGTTTTGCGCTGCTTCGCGATCGAGGA |
| S4-n1 | AAGTCCCCTCGAGTTTTTCCTCCTCGAAGAGGGGGCGGAGAT |
| | CGATTTCCAAAAAACTCGAATGACATCATCTTTCGAAGATCA |
| | AGGGGTCGAAGATCAAAGGGTCGATTTCCAAGAAATCGATTT |
| | CTTGGAAATCGAGGAAAGTCCCCTCGACCCTTTGATCTTCGA |
| | TCTCCGCCCCCTCTTCGAAGATCAAAGGGTCGAAGAGGGGGC |
| | GGAGATCGAGTAGAGTCTAGACTCTACATTTTGACACCCCCA |
| | T |
| SEQ ID NO: 33 | CTGCTTAGGGTTAGGCGTTTTGCGCTGCTTCGCGATCGATTTC |
| S6-n1 | CAAGAAATCGATTTCTTGGAAATCGAATGACATCATCTTTCG |
| | AGTTTTTCCTCCTCGAAGAGGGGGCGGAGATCGAGGAAAGTC |
| | CCCTCGATTTCCAAGAAATCGAACTTCCTTCGAAGGAAGTTC |
| | GAGGAGGAAAAACTCGAGGGGGGGGGTCGAGTTTTTCCTCC |
| | TCGAGGGGACTTTCCTCGACCCCGCCCCCTCGACCCTTTGATC |
| | TTCGATTTCCAAGAAATCGAGTAGAGTCTAGACTCTACATTTT |
| | GACACCCCCAT |
| SEQ ID NO: 34 | TCGAAGATCAAAGGGTCGATTTCCAAGAAATCGATGTGACTC |
| S1-325 | ATTCGATTTGGCGCTCGACCCCGCCCCCTCGAAGAGGGGGCG |
| | GAGATCGAATGAGTCACATCGAGGAAAGTCCCCTCGATTTCT |
| | TGGAAATCGAGGAAAGTCCCCTCGAGGGGACTTTCCTCGAGG |
| | AGGAAAAACTCGAATGACATCATCTTTCGAATGAGTCACATC |
| | GATTTCTTGGAAATCGAGGGGACTTTCCTCGACCCCGCCCCCT |
| | CGAGGTGACTTTCCTCGAGGGGACTTTCCTCGATGTGACTCAT |
| | TCGAGGGGACTTTCCTCGAGGGGACTTTCCTCGACCCTTTGAT |
| | CTTCGATTTGGCGCTCGAGGGGACTTTCCTCGAGGGGACTTTC |
| | CTCGAGGGGACTTTCCTCGAGTAGAGTCTAGACTCTACATTTT |
| | GACACCCCCA |
| SEQ ID NO: 35 | TCGAATGAGTCACATCGATCTCCGCCCCCTCTTCGAGGGGGC |
| S1-60 | GGGGTCGAGGAGGAAAAACTCGAATGAGTCACATCGACCCT |
| | TTGATCTTCGAGGGGACTTTCCGGGGTGGAGCAAGCGTGACA |
| | AGTCCACGTATGACCCGACCGACGATATCGAAGCCTACGCGC |
| | TGAACGCCAGCCCCGATCGACCCCGCCCCCTCGATTTCCAAG |
| | AAATCGAATGACATCATCTTTCGAATGACATCATCTTTCGAG |
| | GGGACTTTCCTCGAACTTCCTTCGAGGGGACTTTCCTCGAGG |
| | GGACTTTCCTCGAGGAGGAAAAACTCGAGTAGAGTCTAGACT |
| | CTACATTTTGACACCCCCA |
| SEQ ID NO: 36 | TCGAATGAGTCACATCGATCTCCGCCCCCTCTTCGAGGGGGC |
| S2-274 | GGGGTCGAGGAGGAAAAACTCGAATGAGTCACATCGACCCT |
| | TTGATCTTCGAGGGGACTTTCCGGGGTGGAGCAAGCGTGACA |
| | AGTCCACGTATGACCCGACCGACGATATCGAAGCCTACGCGC |
| | TGAACGCCAGCCCCGATCGACCCCGCCCCCTCGATTTCCAAG |
| | AAATCGAATGACATCATCTTTCGAATGACATCATCTTTCGAG |
| | GGGACTTTCCTCGAACTTCCTTCGAGGGGACTTTCCTCGAGG |
| | GGACTTTCCTCGAGGGGACTTTCCTCGAGGAGGAAAAACTCG |
| | AAAGATGATGTCATTCGAGTTTTTCCTCCTCGAGGAGGAAAA |
| | ACTCGAGTAGAGTCTAGACTCTACATTTTGACACCCCCA |
| SEQ ID NO: 37 | TCGAATGAGTCACATCGATCTCCGCCCCCTCTTCGAGGGGGC |
| S2-310 | GGGGTCGAGGAGGAAAAACTCGAATGAGTCACATCGACCCT |
| | TTGATCTTCGAGGGGACTTTCCGGGGTGGAGCAAGCGTGACA |
| | AGTCCACGTATGACCCGACCGACGATATCGAAGCCTACGCGC |
| | TGAACGCCAGCCCCGATCGACCCCGCCCCCTCGATTTCCAAG |
| | AAATCGAATGACATCATCTTTCGAATGACATCATCTTTCGAG |
| | GGGACTTTCCTCGAACTTCCTTCGAGGGGACTTTCCTCGAGG |
| | GGACTTTCCTCGAGGGGACTTTCCTCGAGGAGGAAAAACTCG |
| | AGTAGAGTCTAGACTCTACATTTTGACACCCCCA |
| SEQ ID NO: 38 | TCGATTTCCAAGAAATCGACCCCGCCCCCTCGAACTTCCTTCG |
| S1-367 | ATTTCTTGGAAATCGAGGAAAGTCCCCTCGATTTCTTGGAAA |
| | TCGATTTCTTGGAAATCGAATGACATCATCTTTCGATTTCTTG |
| | GAAATCGACCCTTTGATCTTCGAGGAGGAAAAACTCGAATGA |
| | CATCATCTTTCGATCACGTCCTCGAAGATCAAAGGGTCGAGT |
| | TTTTCCTCCTCGAGGAAAGTCCCCTCGATGTGACTCATTCGAT |
| | TTCTTGGAAATCGAGGGGACTTTCCTCGAGGGGACTTTCCTC |
| | GAGTTTTTCCTCCTCGAGGAGGAAAAACTCGATTTCCAAGAA |
| | ATCGAGGGGACTTTCCTCGACCCTTTGATCTTCGAGCGCCAA |
| | ATCGAGTAGAGTCTAGACTCTACATTTTGACACCCCCA |
| SEQ ID NO: 39 | TCGATTTCTTGGAAATCGATTTGGCGCTCGAAGGAAGTTCGA |
| S1-7 | GGGGGCGGGGTCGATTTCCAAGAAATCGAGTTTTTCCTCCTC |
| | GAGCGCCAAATCGACCCTTTGATCTTCGATCACGTCCTCGAG |
| | CGCCAAATCGAGGACGTGATCGAATGAGTCACATCGAATGA |
| | GTCACATCGAATGAGTCACATCGATTTGGCGCTCGATCGGGG |
| | GGCGGGGTCGAGGAAAGTCCCCTCGAGGAAAGTCCCCTCGAT |
| | TTCCAAGAAATCGATTTGGCGCTCGATCTCCGCCCCCTCTTCG |
| | ATCTCCGCCCCCTCTTCGAGTTTTTCCTCCTCGAGTAGAGTCT |
| | AGACTCTACATTTTGACACCCCCA |

### Certain Methods and Compositions for Increasing an Activity of a Cell

Some embodiments of the methods and compositions provided herein include methods for increasing an activity of a cell comprising a chimeric antigen receptor (CAR). Some such methods include introducing into a cell a first nucleic acid encoding a transcription response element (TRE); and a second nucleic acid encoding a CAR, wherein the activity of the cell is increased compared to a cell lacking the first nucleic acid. In some embodiments, the increased activity of the cell is selected from: (i) survival of a subject administered the cell, wherein the subject comprises a target cell comprising an antigen, wherein the CAR is capable of specifically binding to the antigen; (ii) killing of a target cell comprising an antigen, wherein the CAR is capable of specifically binding to the antigen; and (iii) proliferation of the cell in the presence of a target cell comprising an antigen, wherein the CAR is capable of specifically binding to the antigen.

In some embodiments, the TRE comprises a binding site for a TRE of a gene selected from E2F1, EGR1, FOS, HIF1a, JUN, NFAT, LEF1, NFkB, SP1, PU.1, or STAT4.

In some embodiments, the first nucleic acid comprises a nucleotide sequence having at least 80%, 85%, 90%, 91%, 92%, 93%, 94%, 95% 96%, 97%, 98% or 99% sequence identity with any one of SEQ ID NOs: 01-39. In some embodiments, the first nucleic acid comprises the nucleotide sequence of any one of SEQ ID NOs: 01-39. In some embodiments, the first nucleic acid comprises a nucleotide sequence having at least 80%, 85%, 90%, 91%, 92%, 93%, 94%, 95% 96%, 97%, 98% or 99% sequence identity with SEQ ID NO:07. In some embodiments, the first nucleic acid comprises the nucleotide sequence of SEQ ID NO:07.

In some embodiments, the first nucleic acid comprises a minimal promoter linked to the TRE. In some embodiments, the minimal promoter is an IL-2 minimal promoter.

In some embodiments, a polynucleotide comprises the first nucleic acid and the second nucleic acid. In some embodiments, the first nucleic acid is 5′ of the second nucleic acid. In some embodiments, the polynucleotide comprises an insulator located between the first nucleic acid and the second nucleic acid. In some embodiments, the insulator comprises a 3′HS-A insulator. In some embodiments, a vector comprises the polynucleotide. In some embodiments, the vector is a viral vector. In some embodiments, the viral vector is selected from a lentiviral vector, and an adeno-associated viral (AAV) vector.

In some embodiments, a first vector comprises the first nucleic acid, and a second vector comprises the second nucleic acid.

In some embodiments, the CAR comprises a ligand binding domain, a spacer, a transmembrane domain, and an intracellular signaling domain. In some embodiments, the ligand binding domain comprises an antibody or antigen binding fragment thereof. In some embodiments, the ligand binding domain is single chain variable fragment. In some embodiments, the spacer comprises a portion of a hinge region of a human antibody. In some embodiments, the spacer comprises an IgG4 hinge domain. In some embodiments, the intracellular signaling domain comprises all of or a portion of CD3 zeta in combination with a costimulatory domain selected from the group consisting of CD27, CD28, 4-1BB, OX-40, CD30, CD40, PD-1, ICOS, LFA-1, CD2, CD7, NKG2C, B7-H3, and a combination thereof. In some embodiments, the intracellular signaling domain comprises a portion of CD3 zeta and a portion of 4-1BB.

Some embodiments also include stimulating the cell. In some embodiments, the stimulating comprises contacting the cell with an anti-CD3 antibody or antigen binding fragment thereof, an anti-CD28 antibody or antigen binding fragment thereof, or mixture thereof. Some embodiments also include contacting the cell with a compound selected from phorbol 12-myristate 13-acetate (PMA) or Ionomycin.

Some embodiments also include administering the cell to a subject, wherein the subject comprises a tumor cell comprising an antigen, wherein the CAR is capable of specifically binding to the antigen. In some embodiments, the administration comprises an immunotherapy.

In some embodiments, the cell is selected from a T cell, precursor T cell, or a hematopoietic stem cell. In some embodiments, the cell is a CD4+ T cell or a CD8+ T cell. In some embodiments, the cell is a CD8+ T cytotoxic lymphocyte cell selected from the group consisting of a naïve CD8+ T cell, a central memory CD8+ T cell, an effector memory CD8+ T cell, and a bulk CD8+ T cell; or a CD4+ T helper lymphocyte cell selected from the group consisting of a naïve CD4+ T cell, a central memory CD4+ T cell, an effector memory CD4+ T cell, and bulk CD4+ T cell. In some embodiments, the cell is human. In some embodiments, the cell is ex vivo.

Some embodiments of the methods and compositions provided herein include systems for increasing an activity of a cell comprising a chimeric antigen receptor (CAR), comprising: a first nucleic acid encoding a transcription response element (TRE); and a second nucleic acid encoding a CAR, wherein the activity of the cell is increased compared to a cell lacking the first nucleic acid. In some embodiments, the increased activity of the cell is selected from: (i) survival of a subject administered the cell, wherein the subject comprises a target cell comprising an antigen, wherein the CAR is capable of specifically binding to the antigen; (ii) killing of a target cell comprising an antigen, wherein the CAR is capable of specifically binding to the antigen; and (iii) proliferation of the cell in the presence of a target cell comprising an antigen, wherein the CAR is capable of specifically binding to the antigen.

In some embodiments, the TRE comprises a binding site for a TRE of a gene selected from E2F1, EGR1, FOS, HIF1a, JUN, NFAT, LEF1, NFkB, SP1, PU.1, or STAT4.

In some embodiments, the first nucleic acid comprises a nucleotide sequence having at least 80%, 85%, 90%, 91%, 92%, 93%, 94%, 95% 96%, 97%, 98% or 99% sequence identity with any one of SEQ ID NOs: 01-39. In some embodiments, the first nucleic acid comprises the nucleotide sequence of any one of SEQ ID NOs: 01-39. In some embodiments, the first nucleic acid comprises a nucleotide sequence having at least 80%, 85%, 90%, 91%, 92%, 93%, 94%, 95% 96%, 97%, 98% or 99% sequence identity with SEQ ID NO:07. In some embodiments, the first nucleic acid comprises the nucleotide sequence of SEQ ID NO:07.

In some embodiments, the first nucleic acid comprises a minimal promoter linked to the TRE. In some embodiments, the minimal promoter is an IL-2 minimal promoter.

In some embodiments, a polynucleotide comprises the first nucleic acid and the second nucleic acid. In some embodiments, the first nucleic acid is 5′ of the second nucleic acid. In some embodiments, the polynucleotide comprises an insulator located between the first nucleic acid and the second nucleic acid. In some embodiments, the insulator comprises a 3′HS-A insulator. In some embodiments, a vector comprises the polynucleotide. In some embodiments, the vector is a viral vector. In some embodiments, the viral vector is selected from a lentiviral vector, and an adeno-associated viral (AAV) vector.

In some embodiments, a first vector comprises the first nucleic acid, and a second vector comprises the second nucleic acid.

In some embodiments, the CAR comprises a ligand binding domain, a spacer, a transmembrane domain, and an intracellular signaling domain. In some embodiments, the ligand binding domain comprises an antibody or antigen binding fragment thereof. In some embodiments, the ligand binding domain is single chain variable fragment. In some embodiments, the spacer comprises a portion of a hinge region of a human antibody. In some embodiments, the spacer comprises an IgG4 hinge domain. In some embodiments, the intracellular signaling domain comprises all of or a portion of CD3 zeta in combination with a costimulatory domain selected from the group consisting of CD27, CD28, 4-1BB, OX-40, CD30, CD40, PD-1, ICOS, LFA-1, CD2, CD7, NKG2C, B7-H3, and a combination thereof. In some embodiments, the intracellular signaling domain comprises a portion of CD3 zeta and a portion of 4-1BB.

In some embodiments, the cell is in contact with an anti-CD3 antibody or antigen binding fragment thereof, an anti-CD28 antibody or antigen binding fragment thereof, or mixture thereof. In some embodiments, the cell is in contact with a compound selected from phorbol 12-myristate 13-acetate (PMA) or Ionomycin.

In some embodiments, the cell comprises the first nucleic acid and the second nucleic acid. In some embodiments, the cell is selected from a T cell, precursor T cell, or a hematopoietic stem cell. In some embodiments, the cell is a CD4+ T cell or a CD8+ T cell. In some embodiments, the cell is a CD8+ T cytotoxic lymphocyte cell selected from the group consisting of a naïve CD8+ T cell, a central memory CD8+ T cell, an effector memory CD8+ T cell, and a bulk CD8+ T cell; or a CD4+ T helper lymphocyte cell selected from the group consisting of a naïve CD4+ T cell, a central memory CD4+ T cell, an effector memory CD4+ T cell, and bulk CD4+ T cell. In some embodiments, the cell is human. In some embodiments, the cell is ex vivo.

### EXAMPLES

### Example 1-Increased In Vivo Survival with Cells Containing iSynPro

Cells containing an anti-B7H3 CAR and a construct containing an iSynPro (inducible synthetic promoter) had an increased survival against a Be2 tumor challenge in vivo compared to cells lacking the construct containing an iSynPro.

CD4+ T cells and CD8+ T cells were isolated from a blood cone. The CD4+ T cells were transduced with an anti-B7H3 CAR (B7H3CAR), and the CD8 T cells were transduced with either anti-B7H3 CAR, or with the anti-B7H3 CAR and a construct containing an S1-61 iSynPro linked to an IL-2 minimal promoter and a GFP; ffluc reporter (S1-61 IL2mp_GFP; ffluc). Controls included untransduced CD4+ T cells and CD8+ T cells. All T cell lines were transduced with a construct containing an EF1a promoter linked to eGFP; ffluc reporter (EF1a_eGFP; ffluc) to monitor T cell tracking in vivo.

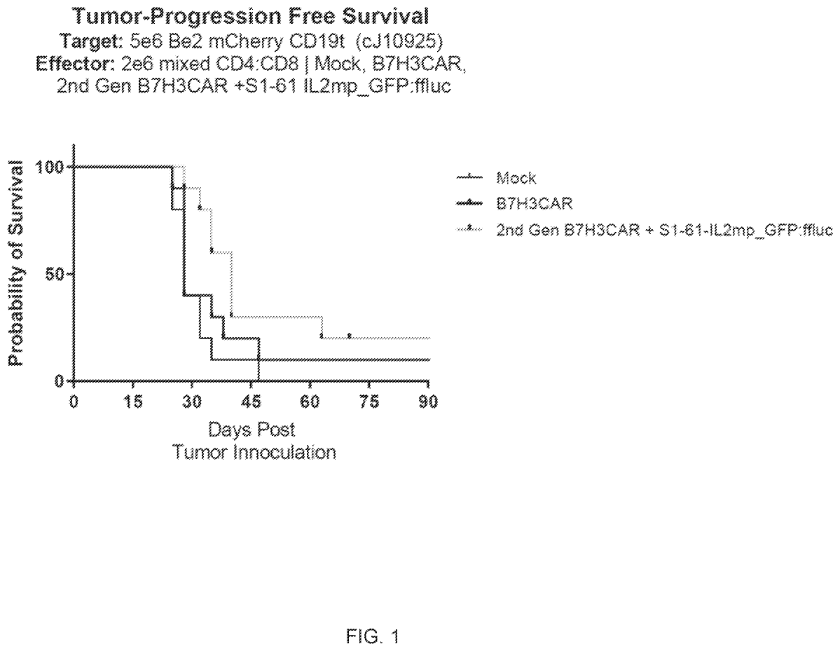

The CD4+ T cells and CD8+ T cells were stimulated with anti-CD3/CD28 beads for 16 days, then expanded using a Seitaro Rapid Expansion protocol for 14 days. Following expansion, cells were cryopreserved and stored in liquid nitrogen. A Be2 neuroblastoma line expressing an mCherry fluorescent marker and a truncated CD19 (CD19t) cell surface marker were expanded and 5e6 cells were injected into the flank of NSG mice. After 5 days, 2e6 cells containing a mixture of the CD4+ T cells and CD8+ T cells were administered by tail vein to the mice. As shown in FIG. 1, mice treated with CD8+ T cells containing the S1-61-IL2mp and the B7H3CAR had an increased survival rate compared to mice treated with B7H3CAR alone.

### Example 2—Extended Killing Capacity of Cells Containing iSynPro

Cells containing a construct encoding an iSynPro and an anti-CD19 CAR had an extended killing capacity over recursive stimulation with Raji target cells in an Incucyte killing assay compared to cells containing a construct encoding an anti-CD19 CAR and lacking an iSynPro sequence.

CD8+ T cells were electroporated using a Lonza 4D to introduce an mCD19CAR construct encoding an anti-CD19 CAR; an huCD19CAR construct encoding an anti-CD19 CAR; a +iGFP: ffluc construct encoding an iSynPro (S1-61) linked to an IL-2 minimal promoter (IL2mp), a GFP: ffluc reporter, an RBG poly A, two 3′HS-A insulators, and an anti-CD19 CAR; or no construct (mock). TABLE 2 lists certain constructs.

TABLE 2

| Construct | Components |
|---|---|
| mCD19CAR | EF1a(s)_CD19scFv(FMC63)-IgG4hinge-CD28tm-41BB-Zeta-T2A-EGFRt- |
| | T2A-DHFRdm_PBNP |
| huCD19CAR | EF1a(L)huCD19scFv(G01S)-IgG4hinge-CD28tm-4-1BB-zeta-P2A- |
| | DHFRdm-T2A-EGFRt_PBNP |
| +iGFP:ffluc | S1-61-IL2mp_GFP:Ffluc_RBGPolyA 2 × (3′HS- |
| | A)_EF1a(7.3)_huCD19scFv(G01S)-IgG4hinge-CD28tm-41BB-zeta-P2A- |
| | DHFRdm-T2A-EGFRt_bGHPolyA_PBNP |

EF1a: EF1a promoter; CD19scFv: anti-CD19 scFV; IgG4hinge: IgG4 hinge spacer; CD28tm: CD29 transmembrane region; 41BB-zeta: 41BB costimulatory domain; T2A: ribosome skip sequence; DHFRdm: dihydrofolate reductase double mutant; S1-61: an iSynPro; IL2mp: IL-2 minimal promoter; GFP:Ffluc: reporter; RBGPolyA: poly-A region; 3′HS-A: insulator; P2A: ribosome skip sequence; bGHPolyA: poly-A region; EGFRt: truncated EGFR polypeptide.

After cell expansion, the CD8+ T cells (effectors) were co-cultured with Raji cells (targets) expressing an mCherry reporter at a 4:1 ratio. The co-culture underwent recursive stimulation by adding 20,000 Raji cells at day 3; and adding 40,000 Raji cells at day 6. mCherry levels were monitored over time by live cell imaging, and mCherry signal was normalized by each respective stimulation.

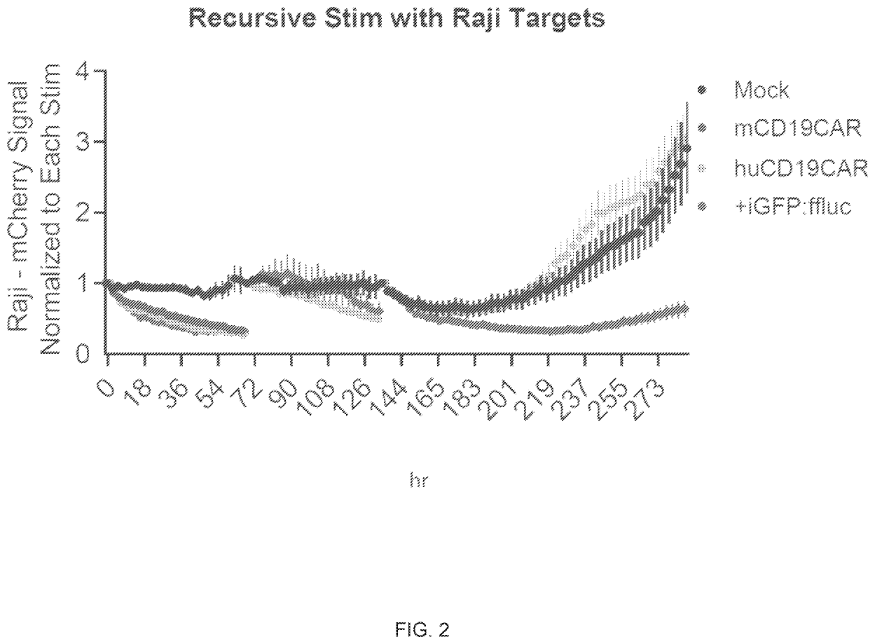

As shown in FIG. 2, CD8+ T cells containing the +iGFP: ffluc construct, which encoded both an iSynPro sequence and an anti-CD19 CAR had an increased killing capacity compared to CD8+ T cells containing mCD19CAR construct or huCD19CAR construct which each encoded an anti-CD19 CAR and lacked an iSynPro sequence.

### Example 3—Enhanced Proliferation of Cells Containing an iSynPro

Cells containing a construct encoding an iSynPro and an anti-CD19 CAR had increased proliferation in the assay of Example 2 compared to cells containing a construct encoding an anti-CD19 CAR and lacking an iSynPro sequence.

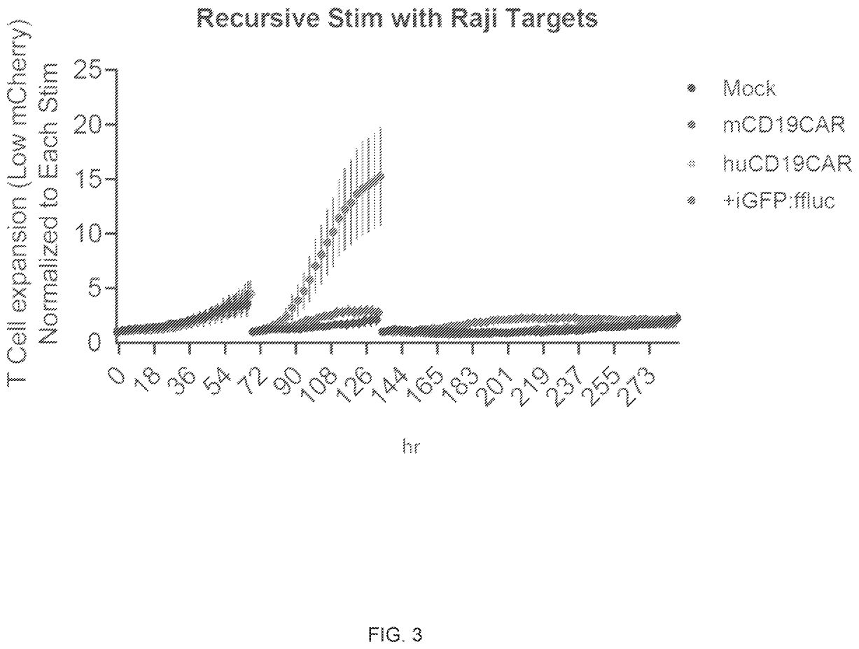

In the assay of Example 2, the number of T cells was tracked by determining the number of cells that lacked mCherry expression (Low mCherry) over time. As shown in FIG. 3, after the second stimulations at day 3, CD8+ T cells containing the +iGFP: ffluc construct which encoded both an iSynPro sequence and an anti-CD19 CAR had increased levels of proliferation compared to CD8+ T cells containing mCD19CAR construct or huCD19CAR construct which each encoded an anti-CD19 CAR and lacked an iSynPro sequence.

### Example 4—Enhanced Tumor Control with Cells Containing iSynPro

CD8+ T cells containing a construct encoding an iSynPro and an anti-CD19 CAR had increased tumor control in a spheroid killing assay compared to cells containing a construct encoding an anti-CD19 CAR and lacking an iSynPro sequence. TABLE 3 lists certain constructs.

TABLE 3

| Transfectent | Construct components |
|---|---|
| cJ13177 | TA Mock |
| cJ13178 | EF1a (L) huCD19scFv(G01S)-IgG4hinge-CD28tm-4-1BB-zeta- |
| | P2A-DHFRdm-T2A-EGFRt_PBNP |
| cJ13179 | S1-61-IL2mp_GFP:ffluc_RBGPolyA_2 × (3′HS- |
| | A)_EF1a(7.3)_huCD19scFv(G01S)-IgG4hinge-CD28tm-41BB-zeta- |
| | P2A-DHFRdm-T2A-EGFRt_bGHPolyA_PBNP |
| cJ13180 | S1-61-IL2mp_GFP:ffluc-T2A-Her2tG_RBGPolyA 2 × (3′HS- |
| | A)_EF1a(7.3)_huCD19scFv(G01S)-IgG4hinge-CD28tm-41BB-zeta- |
| | P2A-DHFRdm-T2A-EGFRt_bGHPolyA_PBNP |
| cJ13181 | S1-61-IL2mp_turboGFP-T2A-Her2tG_RBGPolyA_2 × (3′HS- |
| | A)_EF1a(7.3)_huCD19scFv(G01S)-IgG4hinge-CD28tm-41BB-zeta- |
| | P2A-DHFRdm-T2A-EGFRt_bGHPolyA_PBNP |

EF1a: EF1a promoter; CD19scFv: anti-CD19 scFV; IgG4hinge: IgG4 hinge spacer; CD28tm: CD29 transmembrane region; 41BB-zeta: 41BB costimulatory domain; T2A: ribosome skip sequence; DHFRdm: dihydrofolate reductase double mutant; S1-61: an iSynPro; IL2mp: IL-2 minimal promoter; GFP:ffluc: reporter; RBGPolyA: poly-A region; 3′HS-A: insulator; P2A: ribosome skip sequence; bGHPolyA: poly-A region; EGFRt: truncated EGFR polypeptide; Her2tG: truncated HER2 polypeptide; turboGFP: reporter.

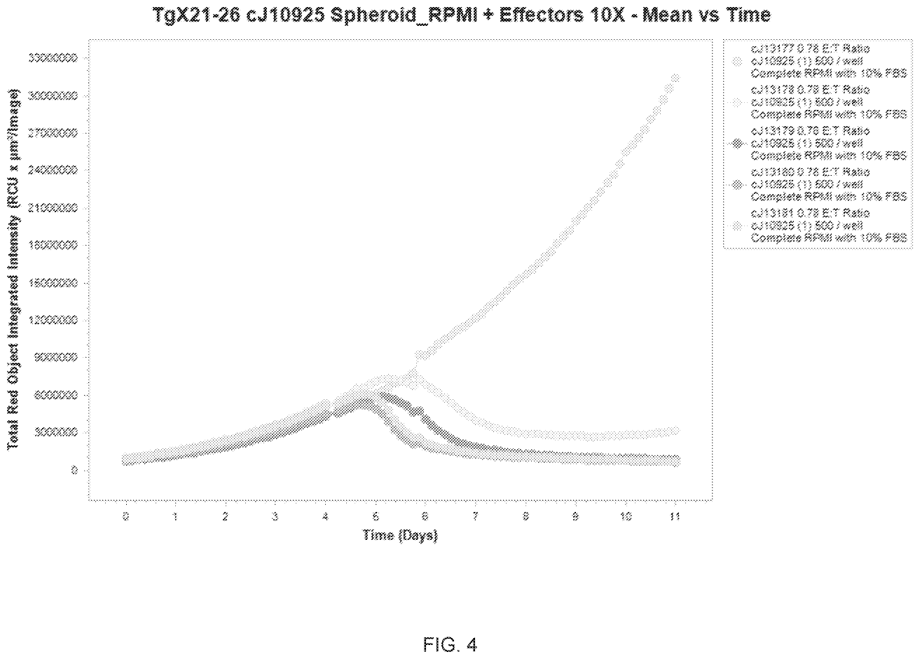

CD8+ T cells containing a construct encoding an iSynPro and an anti-CD19 CAR, or a construct encoding an anti-CD19 CAR and lacking an iSynPro sequence were subjected to a spheroid killing assay. Briefly, human Be2 neuroblastoma cells expressing mCherry and a truncated CD19 (CD19t) polypeptide were plated on ultlalow adherent plates and allowed to form spheroids. After 4 days, the CD8 T cells (effectors) were added to the spheroids (targets) at a E: T ratio of 0.78 to 1 and incubated for a further seven days. The level of mCherry signal was measured using live cell imaging. As shown in FIG. 4, CD8+ T cells containing a construct which encoded both an iSynPro sequence and an anti-CD19 CAR had increased tumor control compared to CD8+ T cells containing a construct, which encoded an anti-CD19 CAR and lacked an iSynPro sequence.

### Example 5—Potency Enhancement Validation by iSynPro

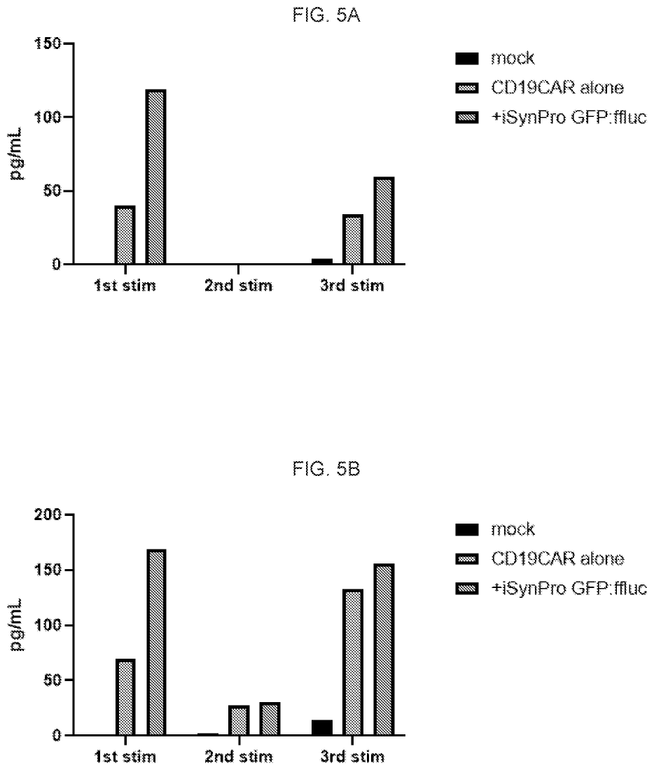

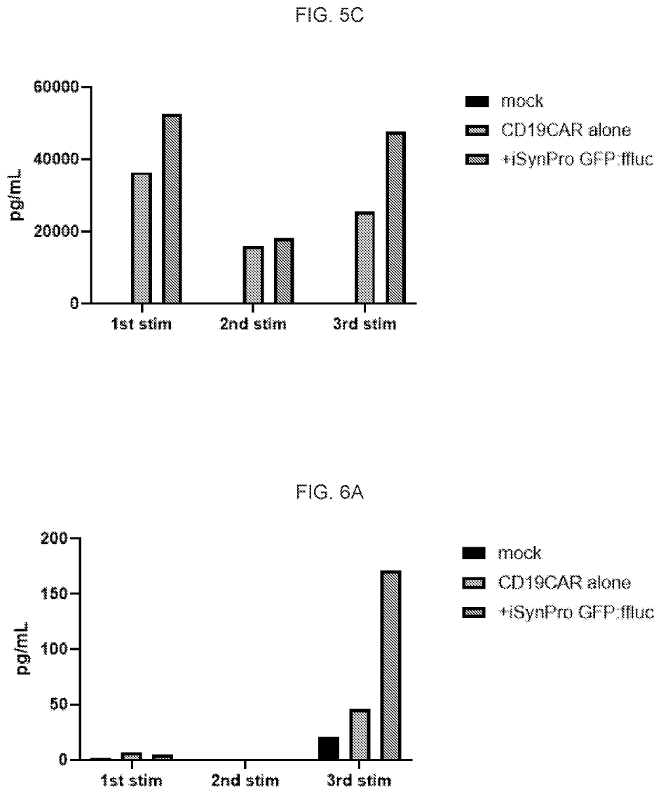

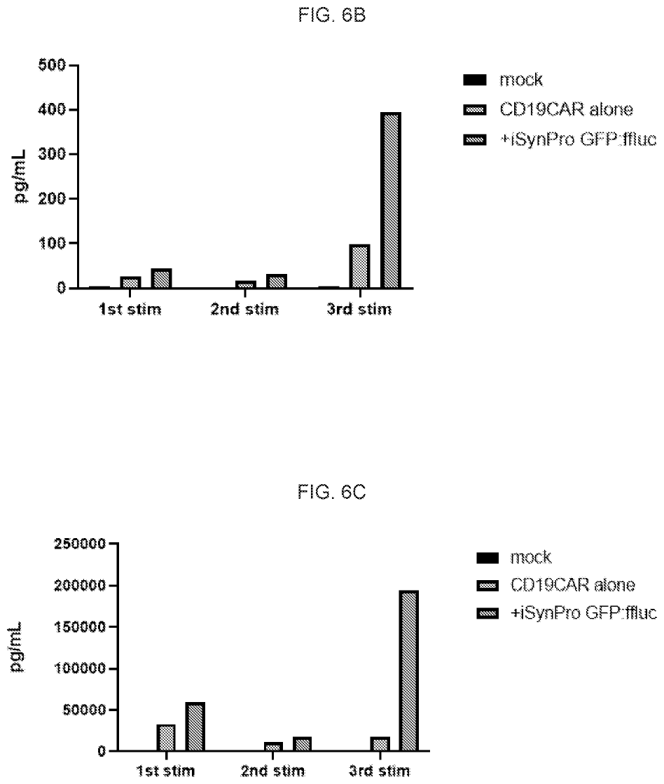

CD4 and CD8 effectors harboring a EF1a_CD19CAR or iSynPro_GFP: ffluc, EF1a_CD19CAR were recursively stimulated for a total of three stimulations, with 72 hours between each stimulation. Cells were stimulated with Be2-CD19t, mCherry targets, and supernatants were collected 24 hours after each stimulation. Cytokine profiles of IL-2, TNF-alpha, and IFN-gamma were determined using the MesoScale Discovery platform (FIGS. 5A-5C and 6A-6C). In CD4s, effectors containing iSynPro and CD19CAR, cytokine production of IL2, TNF-alpha and IFN-gamma were upregulated in response to target stimulation and maintained a higher level of upregulation across the recursive stim assay when compared to EF1a_CD19CAR alone effectors (FIGS. 5A-5C). In CD8s, effectors containing iSynPro_GFP: ffluc, EF1a_CD19CAR maintained a pronounced higher level of expression at the end of the recursive stimulation assay for all three cytokines when compared to CD19CAR alone (FIGS. 6A-6C).

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A method of increasing an activity of a cell comprising a chimeric antigen receptor (CAR), comprising introducing into a cell:
a first nucleic acid encoding a transcription response element (TRE); and
a second nucleic acid encoding a CAR,
wherein the activity of the cell is increased compared to a cell lacking the first nucleic acid.

2. The method of claim 1, wherein the increased activity of the cell is selected from:
(i) survival of a subject administered the cell, wherein the subject comprises a target cell comprising an antigen, wherein the CAR is capable of specifically binding to the antigen;
(ii) killing of a target cell comprising an antigen, wherein the CAR is capable of specifically binding to the antigen; and
(iii) proliferation of the cell in the presence of a target cell comprising an antigen, wherein the CAR is capable of specifically binding to the antigen.

3. The method of claim 1 or 2, wherein the TRE comprises a binding site for a TRE of a gene selected from E2F1, EGR1, FOS, HIF1a, JUN, NFAT, LEF1, NFkB, SP1, PU.1, or STAT4.

4. The method of any one of claims 1-3, wherein the first nucleic acid comprises a nucleotide sequence having at least 95% sequence identity with any one of SEQ ID NOs: 01-39.

5. The method of any one of claims 1-4, wherein the first nucleic acid comprises a nucleotide sequence having at least 95% sequence identity with SEQ ID NO:07.

6. The method of any one of claims 1-5, wherein the first nucleic acid comprises the nucleotide sequence of SEQ ID NO:07.

7. The method of any one of claims 1-6, wherein the first nucleic acid comprises a minimal promoter linked to the TRE.

8. The method of claim 7, wherein the minimal promoter is an IL-2 minimal promoter.

9. The method of any one of claims 1-8, wherein a polynucleotide comprises the first nucleic acid and the second nucleic acid.

10. The method of claim 9, wherein the first nucleic acid is 5′ of the second nucleic acid.

11. The method of claim 8 or 9, wherein the polynucleotide comprises an insulator located between the first nucleic acid and the second nucleic acid.

12. The method of claim 11, wherein the insulator comprises a 3′HS-A insulator.

13. The method of any one of claims 9-12, wherein a vector comprises the polynucleotide.

14. The method of claim 13, wherein the vector is a viral vector.

15. The method of claim 14, wherein the viral vector is selected from a lentiviral vector, and an adeno-associated viral (AAV) vector.

16. The method of any one of claims 1-8, wherein a first vector comprises the first nucleic acid, and a second vector comprises the second nucleic acid.

17. The method of any one of claims 1-16, wherein the CAR comprises a ligand binding domain, a spacer, a transmembrane domain, and an intracellular signaling domain.

18. The method of claim 17, wherein the ligand binding domain comprises an antibody or antigen binding fragment thereof.

19. The method of claim 17 or 18, wherein the ligand binding domain is single chain variable fragment.

20. The method of any one of claims 17-19, wherein the spacer comprises a portion of a hinge region of a human antibody.

21. The method of claim 20, wherein the spacer comprises an IgG4 hinge domain.

22. The method of any one of claims 17-21, wherein the intracellular signaling domain comprises all of or a portion of CD3 zeta in combination with a costimulatory domain selected from the group consisting of CD27, CD28, 4-1BB, OX-40, CD30, CD40, PD-1, ICOS, LFA-1, CD2, CD7, NKG2C, B7-H3, and a combination thereof.

23. The method of any one of claims 17-22, wherein the intracellular signaling domain comprises a portion of CD3 zeta and a portion of 4-1BB.

24. The method of any one of claims 1-23, further comprising stimulating the cell.

25. The method of claim 24, wherein the stimulating comprises contacting the cell with an anti-CD3 antibody or antigen binding fragment thereof, an anti-CD28 antibody or antigen binding fragment thereof, or mixture thereof.

26. The method of any one of claims 1-25, further comprising contacting the cell with a compound selected from phorbol 12-myristate 13-acetate (PMA) or Ionomycin.

27. The method of any one of claims 1-26, further comprising administering the cell to a subject, wherein the subject comprises a tumor cell comprising an antigen, wherein the CAR is capable of specifically binding to the antigen.

28. The method of claim 27, wherein the administration comprises an immunotherapy.

29. The method of any one of claims 1-28, wherein the cell is selected from a T cell, precursor T cell, or a hematopoietic stem cell.

30. The method of any one of claims 1-29, wherein the cell is a CD4+ T cell or a CD8+ T cell.

31. The method of any one of claims 1-30, wherein the cell is a CD8+ T cytotoxic lymphocyte cell selected from the group consisting of a naïve CD8+ T cell, a central memory CD8+ T cell, an effector memory CD8+ T cell, and a bulk CD8+ T cell; or a CD4+ T helper lymphocyte cell selected from the group consisting of a naïve CD4+ T cell, a central memory CD4+ T cell, an effector memory CD4+ T cell, and bulk CD4+ T cell.

32. The method of any one of claims 1-31, wherein the cell is human.

33. The method of any one of claims 1-32, wherein the cell is ex vivo.

34. A system for increasing an activity of a cell comprising a chimeric antigen receptor (CAR), comprising:
a first nucleic acid encoding a transcription response element (TRE); and
a second nucleic acid encoding a CAR,
wherein the activity of the cell is increased compared to a cell lacking the first nucleic acid.

35. The system of claim 34, wherein the increased activity of the cell is selected from:
(i) survival of a subject administered the cell, wherein the subject comprises a target cell comprising an antigen, wherein the CAR is capable of specifically binding to the antigen;
(ii) killing of a target cell comprising an antigen, wherein the CAR is capable of specifically binding to the antigen; and
(iii) proliferation of the cell in the presence of a target cell comprising an antigen, wherein the CAR is capable of specifically binding to the antigen.

36. The system of claim 34 or 35, wherein the TRE comprises a binding site for a TRE of a gene selected from E2F1, EGR1, FOS, HIF1a, JUN, NFAT, LEF1, NFkB, SP1, PU.1, or STAT4.

37. The system of any one of claims 34-36, wherein the first nucleic acid comprises a nucleotide sequence having at least 95% sequence identity with any one of SEQ ID NOs: 01-39.

38. The system of any one of claims 34-37, wherein the first nucleic acid comprises a nucleotide sequence having at least 95% sequence identity with SEQ ID NO:07.

39. The system of any one of claims 34-38, wherein the first nucleic acid comprises the nucleotide sequence of SEQ ID NO:07.

40. The system of any one of claims 34-39, wherein the first nucleic acid comprises a minimal promoter linked to the TRE.

41. The system of claim 40, wherein the minimal promoter is an IL-2 minimal promoter.

42. The system of any one of claims 34-41, wherein a polynucleotide comprises the first nucleic acid and the second nucleic acid.

43. The system of claim 42, wherein the first nucleic acid is 5′ of the second nucleic acid.

44. The system of claim 42 or 43, wherein the polynucleotide comprises an insulator located between the first nucleic acid and the second nucleic acid.

45. The system of claim 44, wherein the insulator comprises a 3′HS-A insulator.

46. The system of any one of claims 42-45, wherein a vector comprises the polynucleotide.

47. The system of claim 46, wherein the vector is a viral vector.

48. The system of claim 47, wherein the viral vector is selected from a lentiviral vector, and an adeno-associated viral (AAV) vector.

49. The system of any one of claims 34-41, wherein a first vector comprises the first nucleic acid, and a second vector comprises the second nucleic acid.

50. The system of any one of claims 34-49, wherein the CAR comprises a ligand binding domain, a spacer, a transmembrane domain, and an intracellular signaling domain.

51. The system of claim 50, wherein the ligand binding domain comprises an antibody or antigen binding fragment thereof.

52. The system of claim 50 or 51, wherein the ligand binding domain is single chain variable fragment.

53. The system of any one of claims 50-52, wherein the spacer comprises a portion of a hinge region of a human antibody.

54. The system of claim 53, wherein the spacer comprises an IgG4 hinge domain.

55. The system of any one of claims 50-54, wherein the intracellular signaling domain comprises all of or a portion of CD3 zeta in combination with a costimulatory domain selected from the group consisting of CD27, CD28, 4-1BB, OX-40, CD30, CD40, PD-1, ICOS, LFA-1, CD2, CD7, NKG2C, B7-H3, and a combination thereof.

56. The system of any one of claims 50-55, wherein the intracellular signaling domain comprises a portion of CD3 zeta and a portion of 4-1BB.

57. The system of any one of claims 34-56, wherein the cell is in contact with an anti-CD3 antibody or antigen binding fragment thereof, an anti-CD28 antibody or antigen binding fragment thereof, or mixture thereof.

58. The system of any one of claims 34-57, wherein the cell is in contact with a compound selected from phorbol 12-myristate 13-acetate (PMA) or Ionomycin.

59. The system of any one of claims 34-58, wherein the cell comprises the first nucleic acid and the second nucleic acid.

60. The system of any one of claims 34-59, wherein the cell is selected from a T cell, precursor T cell, or a hematopoietic stem cell.

61. The system of any one of claims 34-60, wherein the cell is a CD4+ T cell or a CD8+ T cell.

62. The system of any one of claims 34-61, wherein the cell is a CD8+ T cytotoxic lymphocyte cell selected from the group consisting of a naïve CD8+ T cell, a central memory CD8+ T cell, an effector memory CD8+ T cell, and a bulk CD8+ T cell; or a CD4+ T helper lymphocyte cell selected from the group consisting of a naïve CD4+ T cell, a central memory CD4+ T cell, an effector memory CD4+ T cell, and bulk CD4+ T cell.

63. The system of any one of claims 34-62, wherein the cell is human.

64. The system of any one of claims 34-63, wherein the cell is ex vivo.


SEQUENCE LISTING (39 sequences)

SEQ ID NO: 1 - DNA, 265 bp, synthetic construct
  source 1..265: mol_type = other DNA
ctgcttagggttaggcgttttgcgctgcttcgcgatcgaggaggaaaaactcgatgtgactcattcgaagatcaaagggtcgaggaaagtcccctcgaccctttgatcttcgaatgacatcatctttcgaggaaagtcccctcgaggggactttcctcgaggggactttcctcgaatgagtcacatcgatctccgccccctcttcgagcgccaaatcgaggacgtgatcgagtagagtctagactctacattttgacacccccat

SEQ ID NO: 2 - DNA, 232 bp, synthetic construct
  source 1..232: mol_type = other DNA
ctgcttagggttaggcgttttgcgctgcttcgcgatcgagggggcggggtcgatctccgccccctcttcgagtttttcctcctcgaggggactttcctcgaatgacatcatctttcgaccctttgatcttcgaaggaagttcgatcacgtcctcgatttggcgctcgaggggactttcctcgaagatcaaagggtcgagtagagtctagactctacattttgacacccccat

SEQ ID NO: 3 - DNA, 257 bp, synthetic construct
  source 1..257: mol_type = other DNA
ctgcttagggttaggcgttttgcgctgcttcgcgatcgatttccaagaaatcgaggaaagtcccctcgaccctttgatcttcgaggaggaaaaactcgaatgagtcacatcgaccctttgatcttcgaagagggggcggagatcgatctccgccccctcttcgaggaaagtcccctcgaccctttgatcttcgaccccgccccctcgaggaaagtcccctcgagtagagtctagactctacattttgacacccccat

SEQ ID NO: 4 - DNA, 256 bp, synthetic construct
  source 1..256: mol_type = other DNA
ctgcttagggttaggcgttttgcgctgcttcgcgatcgatctccgccccctcttcgaagatcaaagggtcgaggggactttcctcgatttcttggaaatcgaatgacatcatctttcgatttggcgctcgaatgacatcatctttcgaccctttgatcttcgatgtgactcattcgaggggactttcctcgaagatcaaagggtcgaggggactttcctcgagtagagtctagactctacattttgacacccccat

SEQ ID NO: 5 - DNA, 205 bp, synthetic construct
  source 1..205: mol_type = other DNA
ctgcttagggttaggcgttttgcgctgcttcgcgatcgaggggactttcctcgagggggcggggtcgatttggcgctcgatctccgccccctcttcgaatgagtcacatcgaggaaagtcccctcgaggaggaaaaactcgatgtgactcattcgatttccaagaaatcgagtagagtctagactctacattttgacacccccat

SEQ ID NO: 6 - DNA, 283 bp, synthetic construct
  source 1..283: mol_type = other DNA
ctgcttagggttaggcgttttgcgctgcttcgcgatcgatttccaagaaatcgatctccgccccctcttcgaatgacatcatctttcgaatgacatcatctttcgaggaggaaaaactcgaccccgccccctcgatttcttggaaatcgaggaaagtcccctcgagcgccaaatcgaaggaagttcgaatgacatcatctttcgaatgagtcacatcgaggaaagtcccctcgaggaggaaaaactcgagtagagtctagactctacattttgacacccccat

SEQ ID NO: 7 - DNA, 390 bp, synthetic construct
  source 1..390: mol_type = other DNA
ctgcttagggttaggcgttttgcgctgcttcgcgatcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccat

SEQ ID NO: 8 - DNA, 192 bp, synthetic construct
  source 1..192: mol_type = other DNA
ctgcttagggttaggcgttttgcgctgcttcgcgatcgatttccaagaaatcgagtttttcctcctcgagggggcggggtcgaggggactttcctcgaccctttgatcttcgaggaaagtcccctcgagcgccaaatcgatctccgccccctcttcgagtagagtctagactctacattttgacacccccat

SEQ ID NO: 9 - DNA, 283 bp, synthetic construct
  source 1..283: mol_type = other DNA
ctgcttagggttaggcgttttgcgctgcttcgcgatcgaagatcaaagggtcgatttcttggaaatcgatgtgactcattcgatcacgtcctcgaggaggaaaaactcgaggaaagtcccctcgaacttccttcgagggggcggggtcgaatgagtcacatcgaggaaagtcccctcgaggggactttcctcgatttcttggaaatcgaagagggggcggagatcgagtttttcctcctcgaggaaagtcccctcgatcgactctacattttgacacccccat

SEQ ID NO: 10 - DNA, 274 bp, synthetic construct
  source 1..274: mol_type = other DNA
ctgcttagggttaggcgttttgcgctgcttcgcgatcgaatgagtcacatcgaagatcaaagggtcgaccctttgatcttcgatttggcgctcgatgtgactcattcgaccccgccccctcgaggaaagtcccctcgaggaaagtcccctcgaggaaagtcccctcgaggaaagtcccctcgagtttttcctcctcgaagagggggcggagatcgatttggcgctcgaggacgtgatcgagtagagtctagactctacattttgacacccccat

SEQ ID NO: 11 - DNA, 286 bp, synthetic construct
  source 1..286: mol_type = other DNA
ctgcttagggttaggcgttttgcgctgcttcgcgatcgatttccaagaaatcgagtttttcctcctcgatttccaagaaatcgaggggactttcctcgaggggactttcctcgaatgacatcatctttcgatgtgactcattcgaagatcaaagggtcgaaggaagttcgaatgagtcacatcgaatgagtcacatcgacccttgatcttcgaggaggaaaaactcgatttggcgctcgatttggcgctcgagtagagtctagactctacattttgacacccccat

SEQ ID NO: 12 - DNA, 397 bp, synthetic construct
  source 1..397: mol_type = other DNA
ctgcttagggttaggcgttttgcgctgcttcgcgatcgatttcttggaaatcgatttggcgctcgaaggaagttcgagggggcggggtcgatttccaagaaatcgagtttttcctcctcgagcgccaaatcgaccctttgatcttcgatcacgtcctcgagcgccaaatcgaggacgtgatcgaatgagtcacatcgaatgagtcacatcgaatgagtcacatcgatttggcgctcgatcggggggcggggtcgaggaaagtcccctcgaggaaagtcccctcgatttccaagaaatcgatttggcgctcgatctccgccccctcttcgatctccgccccctcttcgagtttttcctcctcgagtagagtctagactctacattttgacacccccat

SEQ ID NO: 13 - DNA, 235 bp, synthetic construct
  source 1..235: mol_type = other DNA
ctgcttagggttaggcgttttgcgctgcttcgcgatcgaatgacatcatctttcgaggaaagtcccctcgaccctttgatcttcgaccccgccccctcgagtttttcctcctcgaggaaagtcccctcgaccctttgatcttcgaggggactttcctcgagggggcggggtcgaggacgtgatcgaggaaagtcccctcgagtagagtctagactctacattttgacacccccat

SEQ ID NO: 14 - DNA, 335 bp, synthetic construct
  source 1..335: mol_type = other DNA
ctgcttagggttaggcgttttgcgctgcttcgcgatcgaagatcaaagggtcgaggaggaaaaactcgaggggactttcctcgaccctttgatcttcgaatgagtcacatcgatttcttggaaatcgaaggaagttcgaggggactttcctcgagtttttcctcctcgaacttccttcgaggaaagtcccctcgaagatcaaagggtcgaaggaagttcgaaagatgatgtcattcgatttcttggaaatcgaagatcaaagggtcgaagagggggcggagatcgatttccaagaaatcgagtagagtctagactctacattttgacacccccat

SEQ ID NO: 15 - DNA, 203 bp, synthetic construct
  source 1..203: mol_type = other DNA
ctgcttagggttaggcgttttgcgctgcttcgcgatcgaatgagtcacatcgagcgccaaatcgaggggactttcctcgagggggcggggtcgagtttttcctcctcgatttccaagaaatcgaggggactttcctcgaggggactttcctcgaggggactttcctcgagtagagtctagactctacattttgacacccccat

SEQ ID NO: 16 - DNA, 287 bp, synthetic construct
  source 1..287: mol_type = other DNA
ctgcttagggttaggcgttttgcgctgcttcgcgatcgagtttttcctcctcgaggggactttcctcgaaagatgatgtcattcgatctccgccccctcttcgaggggactttcctcgaggaaagtcccctcgaggaaagtcccctcgaagatcaaagggtcgaatgagtcacatcgaccctttgatcttcgaccctttgatcttcgatttccaagaaatcgaggaaagtcccctcgaggaaagtcccctcgagtagagtctagactctacattttgacacccccat

SEQ ID NO: 17 - DNA, 293 bp, synthetic construct
  source 1..293: mol_type = other DNA
ctgcttagggttaggcgttttgcgctgcttcgcgatcgaggggactttcctcgaggaaagtcccctcgatttcttggaaatcgatttggcgctcgaggaggaaaaactcgatgtgactcattcgaccctttgatcttcgaggggactttcctcgaggggactttcctcgagcgccaaatcgaggaggaaaaactcgaggggactttcctcgatttcttggaaatcgaaagatgatgtcattcgaccctttgatcttcgagtagagtctagactctacattttgacacccccat

SEQ ID NO: 18 - DNA, 252 bp, synthetic construct
  source 1..252: mol_type = other DNA
ctgcttagggttaggcgttttgcgctgcttcgcgatcgaggaaagtcccctcgaggaaagtcccctcgatttcttggaaatcgaatgacatcatctttcgatcacgtcctcgaggaaagtcccctcgagtttttcctcctcgaggggactttcctcgatttccaagaaatcgatttcttggaaatcgaccctttgatcttcgaggaggaaaaactcgagtagagtctagactctacattttgacacccccat

SEQ ID NO: 19 - DNA, 215 bp, synthetic construct
  source 1..215: mol_type = other DNA
ctgcttagggttaggcgttttgcgctgcttcgcgatcgatgtgactcattcgaggaaagtcccctcgatttggcgctcgatgtgactcattcgatttggcgctcgaggggactttcctcgatttccaagaaatcgaggggactttcctcgaggaaagtcccctcgagtttttcctcctcgagtagagtctagactctacattttgacacccccat

SEQ ID NO: 20 - DNA, 254 bp, synthetic construct
  source 1..254: mol_type = other DNA
ctgcttagggttaggcgttttgcgctgcttcgcgatcgatgtgactcattcgagcgccaaatcgatttccaagaaatcgaatgagtcacatcgaatgacatcatctttcgatgtgactcattcgaggaaagtcccctcgatttcttggaaatcgaggaaagtcccctcgagtttttcctcctcgaagagggggcggagatcgaatgacatcatctttcgagtagagtctagactctacattttgacacccccat

SEQ ID NO: 21 - DNA, 355 bp, synthetic construct
  source 1..355: mol_type = other DNA
ctgcttagggttaggcgttttgcgctgcttcgcgatcgaggggactttcctcgatctccgccccctcttcgaggaaagtcccctcgaaggaagttcgaacttccttcgaggggactttcctcgaaggaagttcgagcgccaaatcgaatgagtcacatcgatttccaagaaatcgatctccgccccctcttcgaggaggaaaaactcgaggggactttcctcgatttccaagaaatcgaggggactttcctcgatcacgtcctcgaggacgtgatcgaatgagtcacatcgatgtgactcattcgaccctttgatcttcgagtagagtctagactctacattttgacacccccat

SEQ ID NO: 22 - DNA, 302 bp, synthetic construct
  source 1..302: mol_type = other DNA
ctgcttagggttaggcgttttgcgctgcttcgcgatcgatttccaagaaatcgagggggcggggtcgaggggactttcctcgaggggactttcctcgaggaaagtcccctcgatctccgccccctcttcgaggaaagtcccctcgaacttccttcgaggaaagtcccctcgaatgagtcacatcgatttggcgctcgaggaaagtcccctcgaggggactttcctcgaggaaagtcccctcgagggggcggggtcgaacttccttcgagtagagtctagactctacattttgacacccccat

SEQ ID NO: 23 - DNA, 192 bp, synthetic construct
  source 1..192: mol_type = other DNA
ctgcttagggttaggcgttttgcgctgcttcgcgatcgaatgagtcacatcgatctccgccccctcttcgatttggcgctcgaggggactttcctcgaccctttgatcttcgaggggactttcctcgaggaaagtcccctcgaggaaagtcccctcgagtagagtctagactctacattttgacacccccat

SEQ ID NO: 24 - DNA, 248 bp, synthetic construct
  source 1..248: mol_type = other DNA
ctgcttagggttaggcgttttgcgctgcttcgcgatcgatttggcgctcgatgtgactcattcgaggaaagtcccctcgaggggactttcctcgaagatcaaagggtcgaaagatgatgtcattcgaggaaagtcccctcgaccccgccccctcgatttcttggaaatcgaatgagtcacatcgatttcttggaaatcgatgtgactcattcgagtagagtctagactctacattttgacacccccat

SEQ ID NO: 25 - DNA, 342 bp, synthetic construct
  source 1..342: mol_type = other DNA
ctgcttagggttaggcgttttgcgctgcttcgcgatcgaggggactttcctcgatgtgactcattcgatttcttggaaatcgatttccaagaaatcgaccccgccccctcgaggaggaaaaactcgatttccaagaaatcgaatgacatcatctttcgaggggactttcctcgaagatcaaagggtcgaagatcaaagggtcgaggaggaaaaactcgatttccaagaaatcgaatgagtcacatcgagtttttcctcctcgatttcttggaaatcgaagagggggcggagatcgatttggcgctcgagtagagtctagactctacattttgacacccccat

SEQ ID NO: 26 - DNA, 268 bp, synthetic construct
  source 1..268: mol_type = other DNA
ctgcttagggttaggcgttttgcgctgcttcgcgatcgaaagatgatgtcattcgaatgagtcacatcgatttggcgctcgagggggcggggtcgatttggcgctcgaaagatgatgtcattcgagtttttcctcctcgaccctttgatcttcgaagagggggcggagatcgaaagatgatgtcattcgaatgagtcacatcgatttccaagaaatcgatttccaagaaatcgagtagagtctagactctacattttgacacccccat

SEQ ID NO: 27 - DNA, 272 bp, synthetic construct
  source 1..272: mol_type = other DNA
ctgcttagggttaggcgttttgcgctgcttcgcgatcgaggaggaaaaactcgatttcttggaaatcgaggggactttcctcgaaagatgatgtcattcgaagatcaaagggtcgatgtgactcattcgaggggactttcctcgagggggcggggtcgaatgacatcatctttcgaggggactttcctcgaggggactttcctcgaatgacatcatctttcgatttcttggaaatcgagtagagtctagactctacattttgacacccccat

SEQ ID NO: 28 - DNA, 223 bp, synthetic construct
  source 1..223: mol_type = other DNA
ctgcttagggttaggcgttttgcgctgcttcgcgatcgatctccgccccctcttcgagtttttcctcctcgatttcttggaaatcgaagatcaaagggtcgatttcttggaaatcgaggggactttcctcgaggaaagtcccctcgaggggactttcctcgaggaggaaaaactcgatttggcgctcgagtagagtctagactctacattttgacacccccat

SEQ ID NO: 29 - DNA, 216 bp, synthetic construct
  source 1..216: mol_type = other DNA
ctgcttagggttaggcgttttgcgctgcttcgcgatcgaaagatgatgtcattcgagtttttcctcctcgaaggaagttcgaggacgtgatcgaagagggggcggagatcgaatgagtcacatcgaaggaagttcgaggaaagtcccctcgaggaaagtcccctcgaagatcaaagggtcgagtagagtctagactctacattttgacacccccat

SEQ ID NO: 30 - DNA, 144 bp, synthetic construct
  source 1..144: mol_type = other DNA
ctgcttagggttaggcgttttgcgctgcttcgcgatcgaccccgccccctcgagcgccaaatcgaggggactttcctcgaggggactttcctcgaggggactttcctcgagtagagtctagactctacattttgacacccccat

SEQ ID NO: 31 - DNA, 235 bp, synthetic construct
  source 1..235: mol_type = other DNA
ctgcttagggttaggcgttttgcgctgcttcgcgatcgagggggcggggtcgaatgacatcatctttcgaggaaagtcccctcgaggaaagtcccctcgaggaaagtcccctcgatttccaagaaatcgaccctttgatcttcgaagatcaaagggtcgatgtgactcattcgaagatcaaagggtcgatttggcgctcgagtagagtctagactctacattttgacacccccat

SEQ ID NO: 32 - DNA, 295 bp, synthetic construct
  source 1..295: mol_type = other DNA
ctgcttagggttaggcgttttgcgctgcttcgcgatcgaggaaagtcccctcgagtttttcctcctcgaagagggggcggagatcgatttccaaaaaactcgaatgacatcatctttcgaagatcaaggggtcgaagatcaaagggtcgatttccaagaaatcgatttcttggaaatcgaggaaagtcccctcgaccctttgatcttcgatctccgccccctcttcgaagatcaaagggtcgaagagggggcggagatcgagtagagtctagactctacattttgacacccccat

SEQ ID NO: 33 - DNA, 308 bp, synthetic construct
  source 1..308: mol_type = other DNA
ctgcttagggttaggcgttttgcgctgcttcgcgatcgatttccaagaaatcgatttcttggaaatcgaatgacatcatctttcgagtttttcctcctcgaagagggggcggagatcgaggaaagtcccctcgatttccaagaaatcgaacttccttcgaaggaagttcgaggaggaaaaactcgagggggcggggtcgagtttttcctcctcgaggggactttcctcgaccccgccccctcgaccctttgatcttcgatttccaagaaatcgagtagagtctagactctacattttgacacccccat

SEQ ID NO: 34 - DNA, 435 bp, synthetic construct
  source 1..435: mol_type = other DNA
tcgaagatcaaagggtcgatttccaagaaatcgatgtgactcattcgatttggcgctcgaccccgccccctcgaagagggggcggagatcgaatgagtcacatcgaggaaagtcccctcgatttcttggaaatcgaggaaagtcccctcgaggggactttcctcgaggaggaaaaactcgaatgacatcatctttcgaatgagtcacatcgatttcttggaaatcgaggggactttcctcgaccccgccccctcgaggtgactttcctcgaggggactttcctcgatgtgactcattcgaggggactttcctcgaggggactttcctcgaccctttgatcttcgatttggcgctcgaggggactttcctcgaggggactttcctcgaggggactttcctcgagtagagtctagactctacattttgacaccccca

SEQ ID NO: 35 - DNA, 354 bp, synthetic construct
  source 1..354: mol_type = other DNA
tcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacaccccca

SEQ ID NO: 36 - DNA, 416 bp, synthetic construct
  source 1..416: mol_type = other DNA
tcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggggactttcctcgaggaggaaaaactcgaaagatgatgtcattcgagtttttcctcctcgaggaggaaaaactcgagtagagtctagactctacattttgacaccccca

SEQ ID NO: 37 - DNA, 369 bp, synthetic construct
  source 1..369: mol_type = other DNA
tcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggggactttcctcgaggaggaaaaactcgagtagagtctagactctacattttgacaccccca

SEQ ID NO: 38 - DNA, 419 bp, synthetic construct
  source 1..419: mol_type = other DNA
tcgatttccaagaaatcgaccccgccccctcgaacttccttcgatttcttggaaatcgaggaaagtcccctcgatttcttggaaatcgatttcttggaaatcgaatgacatcatctttcgatttcttggaaatcgaccctttgatcttcgaggaggaaaaactcgaatgacatcatctttcgatcacgtcctcgaagatcaaagggtcgagtttttcctcctcgaggaaagtcccctcgatgtgactcattcgatttcttggaaatcgaggggactttcctcgaggggactttcctcgagtttttcctcctcgaggaggaaaaactcgatttccaagaaatcgaggggactttcctcgaccctttgatcttcgagcgccaaatcgagtagagtctagactctacattttgacaccccca

SEQ ID NO: 39 - DNA, 361 bp, synthetic construct
  source 1..361: mol_type = other DNA
tcgatttcttggaaatcgatttggcgctcgaaggaagttcgagggggcggggtcgatttccaagaaatcgagtttttcctcctcgagcgccaaatcgaccctttgatcttcgatcacgtcctcgagcgccaaatcgaggacgtgatcgaatgagtcacatcgaatgagtcacatcgaatgagtcacatcgatttggcgctcgatcggggggcggggtcgaggaaagtcccctcgaggaaagtcccctcgatttccaagaaatcgatttggcgctcgatctccgccccctcttcgatctccgccccctcttcgagtttttcctcctcgagtagagtctagactctacattttgacaccccca